(19) United States
(12) Patent Application Publication
(10) Pub. No.: US 2014/0012120 A1
(43) Pub. Date: Jan. 9, 2014

(54) HEMOSTASIS SENSOR AND METHOD OF USE THEREOF

(71) Applicant: Accumed Radial Systems, LLC, Ann Arbor, MI (US)

(72) Inventors: Mauricio Gabriel Cohen, Miami, FL (US); James Patrick Ryan, Wilton, NH (US); James G. Downward, IV, Ann Arbor, MI (US); Joseph R. Korotko, Livonia, MI (US)

(73) Assignee: Accumed Radial Systems, LLC, Ann Arbor, MI (US)

(21) Appl. No.: 14/016,034

(22) Filed: Aug. 30, 2013

Related U.S. Application Data
(63) Continuation-in-part of application No. 13/769,733, filed on Feb. 18, 2013.
(60) Provisional application No. 61/634,772, filed on Mar. 6, 2012.
(60) Provisional application No. 61/695,291, filed on Aug. 30, 2012.

(51) Int. Cl. A61B 5/02; A61B 5/00; A61B 8/08

(52) U.S. Cl. CPC A61B 5/02042; A61B 8/488; A61B 5/6831
USPC 600/371

(57) ABSTRACT

An apparatus and method for capturing sensor readings relating to one or more blood flow attributes of a patient at or around a puncture site when the patient is being subjected to hemostasis process by a health care provider. A wide range of different sensor technologies and component configurations can be utilized. The apparatus can operate as a fully stand-alone device, a fully integrated component of a hemostasis treatment tool, or any configuration between the two polar extremes of integration/stand-alone.

### RELATED APPLICATIONS

This utility patent application claims priority to the following patent applications which are hereby incorporated by reference in their entirety: (1) the provisional patent application titled “FLEXIBLE HOLOGRAM USING ROOM LIGHTS WHICH DETECTS ARTERY PULSATION” (Ser. No. 61/634,772) filed on Mar. 6, 2012; (2) the provisional patent application titled “HEMOSTASIS APPARATUS AND METHOD” (Ser. No. 61/695,291) filed on Aug. 30, 2012; and (3) the utility patent application titled “HEMOSTASIS APPARATUS AND METHOD” (Ser. No. 13/769,733) filed on Feb. 18, 2013.

### BACKGROUND OF THE INVENTION

The invention is a sensor for use in the process of performing hemostasis on patients (the “hemostasis sensor”, “sensor apparatus”, or simply the “apparatus” or “sensor”). More specifically, the sensor can provide for the capture of information relating to the flow of blood in the patient (“sensor readings”) as hemostasis is performed on the patient. The sensor can be implemented as a stand-alone apparatus, an apparatus that is used in conjunction with other devices in a non-integrated manner, or as a component integrated into another hemostasis device, such as a hemostasis band used to apply pressure on the site of the bleeding (the “puncture site”).

I. Hemostasis

Hemostasis means the “stoppage of bleeding or hemorrhage”. Human beings and other animals require the flow of blood to sustain life. Blood loss can be fatal to a patient, but steps taken to stem the loss of blood in a patient can also negatively impact the flow of blood in the patient. Thus, hemostasis can be a delicate process by which doctors and other healthcare professionals must navigate between the two extremes of (1) insufficient pressure on the puncture site to stop the bleed (resulting in continued bleeding and blood loss); and (2) too much pressure being applied to the puncture site such that blood flow in the patient is constricted (resulting in harm to the patient from inadequate blood flow).

II. Different Causes of Bleeding

There are many contexts in the providing of healthcare to patients when it is necessary to address bleeding or hemorrhage of a patient. Regardless of the cause of the bleeding, information about the flow of blood through and around the puncture site can be highly useful to providers in the treatment of patients undergoing hemostasis.

A. Bleeding that Results from a Patient Condition

In many instances, bleeding is the result of a medical condition of the patient. Examples of bleeding caused by the medical condition of a patient can include diseases, disorders, injuries caused by accidents, allergies, and other conditions that providers seek to address (collectively “conditions”).

B. Bleeding that Results from Medical Treatment

Bleeding can also result from the providing of medical treatment and/or diagnosis of the patient. Whether the healthcare activity is undertaken for the purposes of diagnosis (such as a blood test) or treatment (such as the injection of medicine into the patient), activities performed by providers (collectively “treatment”) can result in bleeding that must be addressed.

C. Intravascular Catheterizations

Intravascular catheterization includes the catheterization of either the arterial or venous systems for diagnosis or treatment of diseases for all regions of the body, such as cardiovascular, neural (brain), pulmonary (lungs), renal (kidneys) and peripheries. Cardiac catheterization is a subset of intravascular catheterization used to diagnose and treat heart conditions. According the Centers for Disease Control and Prevention, heart disease is the leading cause of death in the United States. Cardiac catheterization involves inserting small tubes (“catheters”) into the circulatory system of the patient. Using X-ray guidance and other sensors, information about blood flow and blood pressure is obtained. Dyes can be injected into the circulatory system for the purpose of identifying the existence of obstructions such as atherosclerotic plaque within blood vessels. On the basis of the location and number of obstructions, a treatment plan for the patient is devised. Such a treatment plan can utilize different devices, specialized medications, placement of a stent to maintain vessel patency and/or bypass surgery.

At the beginning of the catheterization procedure a provider 92 will puncture the vessel to gain access. After gaining access, the necessary catheters are inserted through the ‘access site’ or ‘puncture site’. At the end of the catheterization procedure when all the catheters are removed, the puncture site must be properly closed. A conventional bandage is insufficient because an artery will bleed out through the bandage because it cannot apply sufficient pressure. The proper amount of pressure, or force, needs to be applied at the puncture site to stop bleeding. The pressure can be applied manually by a health care professional holding pressure with their hand, or a medical device (apparatus) can be used to apply pressure.

Cardiac catheterization and other types of intravascular catheterization are commonly performed through either puncture the femoral artery in the groin (“femoral catheterization”) or the radial artery in the wrist (“radial catheterization”).

1. Femoral Catheterization

Femoral catheterization has traditionally been the more common catheterization because the femoral artery is large and the femoral artery provides a direct route to the heart. However, femoral catheterization can require the patient to lie flat without bending their leg for between 2-6 hours during recovery. In some cases, internal bleeding can occur with femoral catheterization even when the patient fully complies with the immobility restrictions.

2. Radial Catheterization

Radial catheterization involves a puncture site located on the radial artery. Radial catheterization has many advantages to femoral catheterization. The radial artery is smaller than the femoral artery and is closer to the surface of the skin than the femoral artery. Thus, internal bleeding can be avoided and the likelihood of complications reduced. Unlike with femoral catheterization, radial catheterization does not require the patient to be immobile. Moreover, patients find radial catheterization to be the more comfortable option because they are free to sit up, walk around, and even eat.

III. Omissions/Risks not Addressed by the Prior Art

The term “hemostasis” refers to the objective of stopping bleeding. Hemostasis is achieved by the application of sufficient pressure at the puncture site to stop the bleeding. The term “patency” refers to the objective of providing for the unobstructed flow of blood in the patient. “Patency” is achieved by avoiding the application of too much pressure to the puncture site, such that blood flow would be impeded. To achieve both “hemostasis” and “patency” requires the successful avoidance of two extremes—insufficient pressure and too much pressure.

Conventional hemostasis tools do not provide a way to monitor blood flow at or around the puncture site as hemostasis is performed, and thus the prior art fails to even address the issue of “patency” in the context of performing hemostasis. The prior art does not provide a convenient way to tell providers that too much pressure is being applied to the puncture site, and that blood flow around the puncture side is being impeded. Prior art tools fail to provide a way for doctors or other health care providers to determine whether the desired magnitude of pressure is applied or not. Prior art tools fail to provide providers with an alert pertaining to the application of too much pressure resulting in impeded blood flow.

The failure of conventional tools to provide such information is a missed opportunity to selectively adjust the pressure applied to the puncture site.

### SUMMARY OF THE INVENTION

The invention is a sensor for use in the process of performing hemostasis on patients (the “hemostasis sensor”, “sensor apparatus”, or simply the “apparatus” or “sensor”). More specifically, the sensor can provide for the capture of information relating to the flow of blood in the patient (“sensor readings”) as hemostasis is performed on the patient.

The sensor apparatus can be applied in a wide variety of different embodiments and configurations. For example, the sensor can be implemented as: (1) a stand-alone device; (2) a tool designed to work in conjunction with other separate hemostasis treatment devices (such as a hemostasis band) in a non-integrated manner; or (3) a component that is fully integrated into a hemostasis treatment device.

In some embodiments of the sensor, the sensor will capture sensor readings and then utilize a separate communication component to communicate the sensor readings to the health care providers. In other embodiments of the sensor, the functionality of the sensor and of the communication component is integrated into a single integrated and indivisible component.

The sensor apparatus detects attributes relating to the blood flow (“blood flow attributes” or simply “blood flow”) at or around the area of the puncture site for the purpose of indicating “patency” to the provider while “hemostasis” is being performed on the patient. Blood flow attributes can be detected directly or indirectly, depending on the particular embodiment of sensor. For example, some types of sensor can detect the pulsatile wave that results from the flow of blood at or around the puncture site. Other embodiments may measure different types of blood flow attributes.

A wide variety of different types and categories of sensor technology can be used to capture sensor readings relating to patient blood flow. Sensors can be electronic sensors (including for example Doppler sensors and ultrasound sensors), mechanical sensors (typically based on the mechanics of pulsing blood flow), electro-mechanical sensors, and thin films that are referred to as “visual pulse indicators” in the patent application titled “HEMOSTASIS APPARATUS AND METHOD” on Feb. 18, 2013 and associated with Ser. No. 13/769,733) and referred to as a hologram in the patent application tiled “FLEXIBLE HOLOGRAM USING ROOM LIGHTS WHICH DETECTS ARTERY PULSATION” (Ser. No. 61/634,772) filed on Mar. 6, 2012.

### BRIEF DESCRIPTION OF THE DRAWINGS

Many features and inventive aspects of the hemostasis sensor apparatus (“hemostasis apparatus”, “sensor apparatus” or simply the “apparatus”) are illustrated in the following drawings:

FIG. 1a is a block diagram illustrating an example of interaction between a patient and healthcare provider using the sensor apparatus and different components of the apparatus, such as a sensor component for obtaining a blood flow attribute from a patient and a communication component for making a sensor reading accessible to a provider.

FIG. 1b is a flow chart diagram illustrating an example of a process for utilizing a hemostasis sensor apparatus in order to allow a provider to adjust the pressure applied at the puncture site of the patient in order to sustain proper blood flow at and around the puncture site.

FIG. 1c is an environmental diagram illustrating an example of a puncture site involving the radial artery during a radial catheterization procedure.

FIG. 1d is an input/output diagram illustrating an example of a hemostasis sensor apparatus, and corresponding input parameters (i.e. patient blood flow attributes) and output indicators/indications (i.e. sensor readings).

FIG. 1e is a block diagram illustrating an example of the hemostasis sensor apparatus interacting with an exterior information technology system.

FIG. 2a is a hierarchy diagram illustrating an example of different categories and subcategories of sensors, such as electronic sensors (including but not limited to Doppler sensors and ultrasound sensors), mechanical sensors (including but not limited to piston assemblies and pulse gauges), electro-mechanical sensors, and films (including but not limited to reflective foils).

FIG. 2b is a block diagram illustrating an example of various optional components that can be included in a hemostasis sensor apparatus, with such optional components including a body component, a timer component, a fastening component, a pressure component, a padding component, a sensor enhancement component, an adhesive component, and anti-adhesion component, a computer, and a transmitter.

FIG. 3a is diagram illustrating an example of a top-view of a film as a sensor component.

FIG. 3b is a diagram illustrating an example of a side-view of the diagram in FIG. 3a.

FIG. 3c is diagram illustrating an example of a top-view of a reflective foil as a sensor component.

FIG. 3d is a diagram illustrating an example of a side-view of the diagram in FIG. 3c.

FIG. 3e is diagram illustrating an example of a top-view of a film with a pattern such as Moiré line pattern.

FIG. 3f is a diagram illustrating an example of a side-view of the diagram in FIG. 3e.

FIG. 3g is a diagram illustrating an example of a top-view of a film (an example of a sensor component) encased in a sheath.

FIG. 3h is a diagram illustrating an example of a side-view of a film (an example of a sensor component) encased in a sheath.

FIG. 3i is a diagram illustrating an example of an alternate side-view of a film (an example of a sensor component) encased in sheath.

FIG. 3j is a diagram illustrating an example of a side-view of a film (an example of a sensor component) encased in a sheath with an anti-adhesion layer positioned between the sheath and the patient.

FIG. 3k is a diagram showing the view of FIG. 3j turned 90 degrees.

FIG. 3l is a diagram illustrating an example of a top view of a film that includes a window for viewing the puncture site.

FIG. 3m is a diagram illustrating an example of a top view of a film that is comprised of a grid that includes 4 zones.

FIG. 4 is a diagram illustrating an example of a piston assembly.

FIG. 5a is diagram illustrating an example of an electronic timer, which is an example of a timing component.

FIG. 5b is a diagram illustrating an example of an ink strip, which is an example of a timing component.

FIG. 5c is a diagram illustrating an example of a balloon that can function as a sensor enhancement component.

FIG. 5d is a diagram illustrating the balloon of FIG. 5c that can function as a sensor enhancement component and can work in conjunction with a sensor enhancement component in the form a “middle member” or “air pocket”.

FIG. 5e is a diagram illustrating an example of sensor apparatus that includes a reflective foil, a window, a balloon, an inlet for the balloon, and two gaps to facilitate movement along a band or body.

FIG. 5f is a diagram illustrating an example of a strip, which is a subcategory of body components.

FIG. 5g is a diagram illustrating an example of a loop, which is a subcategory of body components.

FIG. 5h is a diagram illustrating an example of a semi-flexible/semi-rigid band, which is a subcategory of body components.

FIG. 5i is a diagram illustrating an example of a pad, which is a subcategory of padding components.

FIG. 5j is a diagram illustrating an example of a balloon that can function as a padding component.

FIG. 6 is a diagram illustrating an example of an apparatus that includes a pressure gauge as both a sensor component and a communication component.

FIG. 7 is a diagram illustrating an example of a sensor apparatus that is neither a fully integrated hemostasis band nor a stand-alone sensor.

FIG. 8 is a diagram illustrating an example of sensor apparatus without a body component.

FIG. 9a is a diagram illustrating a front view of a hemostasis band that includes a body component, a padding component, a fastener component, a pressure component, and that a variety of different sensor components and communication components could be added to as an integrated device.

FIG. 9b is a diagram illustrating an example of perspective view of the hemostasis band illustrated in FIG. 9a.

FIG. 9c is a diagram illustrating an example of a side view of the hemostasis band illustrated in FIG. 9a.

FIG. 10a is a diagram illustrating an example of a front view of hemostasis band with a film as a sensor component.

FIG. 10b is a diagram illustrating an example of aside view of the hemostasis band illustrated in FIG. 10a.

FIG. 10c is a diagram illustrating an example of a side view of the hemostasis band illustrated in FIG. 10a, also illustrating an example of an adhesive component between the pressure component and the film.

FIG. 10d is a diagram illustrating a close-up detailed view of a portion of FIG. 10c, but utilizing a non-adhesion component between the film and the radial artery of the patient.

FIG. 11a is a diagram illustrating an example of a side view of a sensor apparatus embodied in an integrated hemostasis band.

FIG. 11b is a diagram illustrating an example of a perspective view of the apparatus illustrated in FIG. 11a.

FIG. 11c is diagram illustrating an example of the apparatus of FIGS. 11a and 11b, but from a different perspective view.

FIG. 11d is a diagram illustrating an example of front view of the apparatus illustrated in FIGS. 11a-11c.

FIG. 11e is a diagram illustrating an example of a side view of the apparatus illustrated in FIGS. 11a-11d, but in a closed position.

FIG. 11f is a diagram illustrating an example of a perspective view of the apparatus illustrated in FIG. 11e.

FIG. 12 is a flow chart diagram illustrating an example of a process for using a hemostasis sensor apparatus in performing hemostasis on a patient.

### DETAILED DESCRIPTION

The invention is a sensor for use in the process of performing hemostasis on patients (the “hemostasis sensor”, “sensor apparatus”, or simply the “apparatus” or “sensor”). More specifically, the sensor can provide for the capture of information relating to the flow of blood in the patient (“sensor readings”) as hemostasis is performed on the patient.

I. Overview

The sensor apparatus provides for the monitoring of information related to a patient's blood flow at or around the puncture site (collectively “blood flow attributes” or simply “blood flow”) of the patient while pressure is applied at the puncture site to stop the bleeding from the puncture site. Healthcare providers can adjust the pressure placed on the puncture site during hemostasis in response to information captured by the sensor apparatus that pertains to blood flow at or around the puncture site of the patient. Use of the sensor apparatus can assist health care providers to avoid the application of excessive pressure on the puncture site of a patient.

A. Elements/Components View

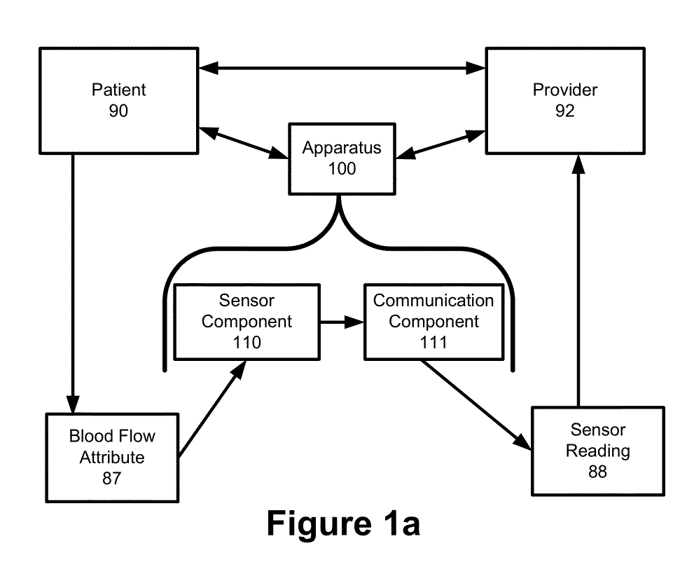

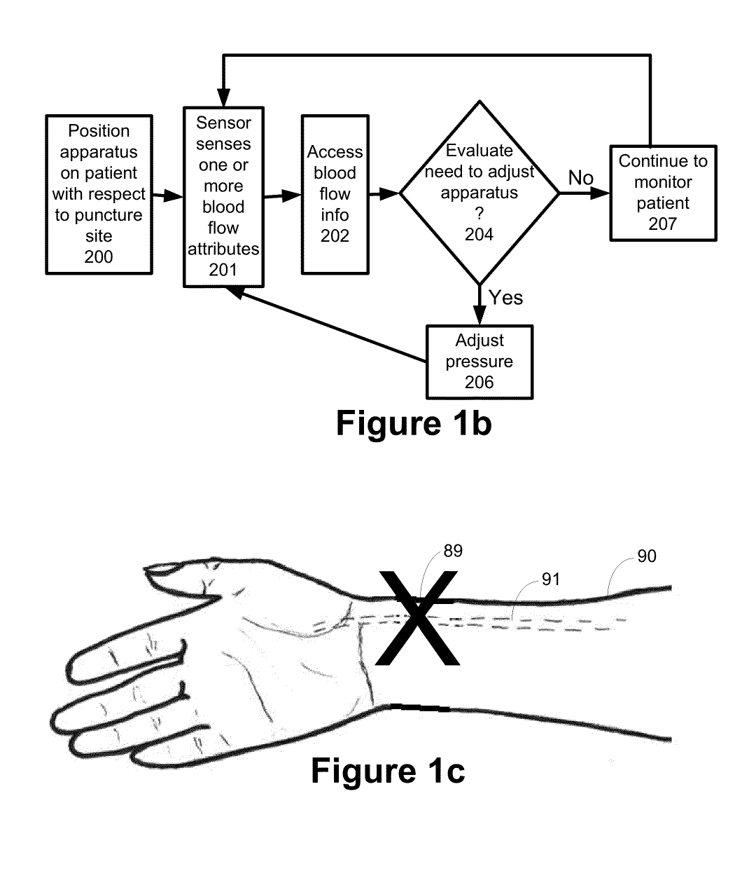

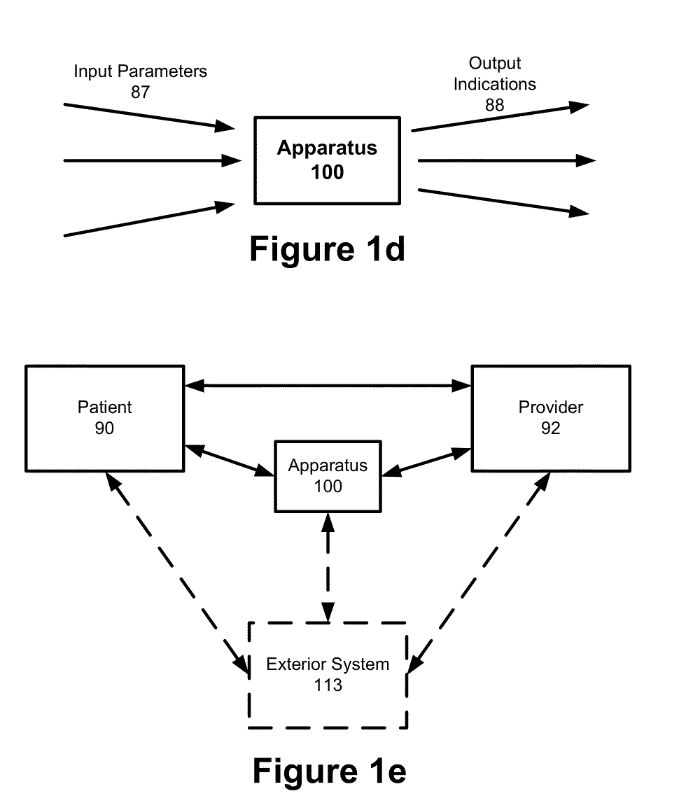

FIG. 1a is a block diagram illustrating an example of a hemostasis sensor apparatus and method (a “sensor apparatus” 100 or simply the “apparatus” 100). The apparatus 100 is used as part of the interactions between a patient 90 and a healthcare provider 92 during hemostasis, a process in which a patient's 90 bleeding is addressed by one or more providers 92 through the application of pressure at the puncture site. Both providers 90 and patients 92 interact with each other during the hemostasis process, and both providers 90 and patients 92 interact with the apparatus 100 as part of the hemostasis process.

As illustrated in FIG. 1a, the apparatus 100 has two subcomponents, a sensor component 110 that provides for the capture of a blood flow attribute 87 (i.e. potentially any information that directly or indirectly relates to whether there is proper blood flow) from the patient 90 and a communication component 111 that provides the provider 92 with access to a sensor reading 88 that corresponds to the blood flow attribute 87 sensed by the sensor component 110.

B. Process Flow View

FIG. 1b is a flow chart diagram illustrating an example of a method for using a hemostasis sensor apparatus 100. At 200, the apparatus 100 is positioned relative to the patient 90 with respect to a puncture site. At 201, the apparatus 100 senses (through use of one or more sensor components 110) one or more blood flow attributes 87 relating to the patient 90 at or around the puncture site. At 202, the provider 92 accesses the blood flow information in the form of one or more sensor readings 88 made accessible to the provider by one or more communication components 111 within the sensor apparatus 100. At 204 the provider 92 makes an evaluation regarding the patient 90 based on the pulse/blood flow information embodied in the one or more sensor readings 88 made accessible to the provider 92. If the sensor readings 88 at 204 suggest that blood flow is being impeded, the device or apparatus applying the pressure can be adjusted at 206 to enhance the blood flow of the patient 90. If the sensor readings 88 at 204 suggest that there is proper and sufficient blood flow, then at 207 no changes are made and the patient 90 continues to be monitored throughout the hemostasis process. The loop from 201 through either 206 or 207 and back again can repeat itself until the hemostasis process is complete.

C. Operating Contexts

The sensor apparatus 100 can be used in the context of radial catheterization, femoral catheterization, or in addressing the bleeding resulting from many different medical conditions and treatments/procedures of the patient 90. Many different contexts of attempting to stop the bleeding of a patient 90 can benefit from the ability to accurately sense the adequacy of pulse or blood flow at or around the puncture site of the patient 90.

However, the original inspiration for the conception of the sensor apparatus 100 did occur in the context of radial catheterization.

FIG. 1c is an environmental diagram illustrating an example of some relevant elements and locations on a patient 90 undergoing radial catheterization. Illustrated in FIG. 1c is an example of an arm of a patient 90. Running through that arm is a radial artery 91. The radial artery 91 is beneath the skin of the patient 90. A typical puncture site 89 for radial catheterization is below the wrist of the hand of the patient 90.

D. Input Parameters/Output Indications

The sensor apparatus 100 is a mechanism by which information relating to the patient 90 is sensed by the sensor apparatus 100 during the hemostasis process, and made accessible to the provider 92 while hemostasis is being performed on the patient 90.

FIG. 1d is an input/output diagram illustrating the concept that sensor apparatus 100 can generate one or more output indicators/indications 88 (which can also be referred to as sensor readings 88) from one or more input parameters 87 (which can also be referred to as blood flow attributes 87). Pulse or blood flow information relating to the area at or around the puncture site 89 is one example of such information. The sensor apparatus 100 can however be used to capture and convey a wider range of information, depending on the particular embodiment of the apparatus 100.

E. Integration with Exterior Systems

FIG. 1e is a block diagram that is similar in many respects to FIG. 1a in that it illustrates the use of the apparatus 100 in the context of a hemostasis process that includes the patient 90, the provider 92, and the apparatus 100. Unlike FIG. 1a, FIG. 1e also discloses an optional element, the element of a exterior information technology system 113 used to integrate data relating to the patient 90, or to the providing of healthcare to the patient 90.

For example, the sensor readings 88 relating to the blood flow of the patient 90 may be useful to other automated, manual, or partially automated/partially manual systems used to manage the health of the patient 90 and the delivery of healthcare to the patient 90 by one or more providers 92. By way of further and different example, there could be a range of different medical conditions or personal attributes relating to the patient 90 that could impact the determination of whether the hemostasis process for the particular patient 90 was proceeding appropriately. Information captured from the sensor component 110 could be used in conjunction with data from other sources to make the determination. In some embodiments, the exterior system 113 can implement maximum data integration to provide for various heuristics tailoring care to the specific requirements and conditions of the patient 90.

II. Alternative Embodiments

No patent application can expressly disclose all of the potential embodiments of an invention. In accordance with the provisions of the patent statutes, the principles and modes of operation of the sensor apparatus 100 are explained and illustrated in certain embodiments. However, it must be understood that the apparatus 100 may be practiced otherwise than is specifically explained and illustrated without departing from its spirit or scope. For example, a wide variety of different potential sensor technologies can be employed in an effort to determine whether there is proper blood flow at and around the puncture point of the patient. Mechanical sensors, electronic sensors, electro-mechanical sensors, and materials such as reflective foil and other materials can perform the function of capturing and conveying information about the pulse or blood flow of the patient. By way of further example, such sensors can be organized into a wide variety of different operational configurations ranging from permanent integration into a hemostasis device such as a hemostasis band to a fully stand alone device, and to every possibility in between those two polar opposites.

The description of the apparatus provided below should be understood to include all novel and non-obvious combination of elements described herein, and claims may be presented in this or a later application to any novel non-obvious combination of these elements. Moreover, the foregoing embodiments are illustrative, and no single feature or element is essential to all possible combinations that may be claimed in this or a later application.

III. Introduction of Elements

FIG. 1a is a block diagram illustrating an example of interaction between a patient 90 and healthcare provider 92 using the sensor apparatus 100, and different components of the apparatus 100, such as a sensor component 110 for obtaining a blood flow attribute 87 from a patient 90 and a communication component 111 for making a sensor reading 88 accessible to a provider 92.

The apparatus 100 is a device used in the treatment of patients 90.

A. Patient

A patient 90 is typically a human being, although the apparatus 100 (or alternative variations thereof) can also be used in the treatment of potentially any organism capable of bleeding.

In the context of human beings, patients 90 can vary widely in terms of age, size, gender, weight, medical status, and other relevant attributes. The original inspiration leading to the conception of the apparatus 100 was for the apparatus 100 to be used in the context of cardiac catheterization. In particular, the apparatus 100 may be particularly useful in the context of radial catheterizations. However, the apparatus 100 (or alternative variations thereof) can also be used in a variety of contexts that involve cardiovascular care and the treatment of wholly different conditions.

In some instances and embodiments of the apparatus 100, the patient 90 can apply and utilize the apparatus 100 without the assistance of a healthcare provider 92 such as a physician, physician's assistant, nurse, paramedic, or other form of caregiver. However, in many instances, the apparatus 100 will be used in the context of a potentially broad range of interactions between the patient 90 and the provider 92.

B. Provider

A provider 92 is typically a healthcare professional such as a physician, nurse, nurse practitioner, catheterization lab technician, physician assistant, paramedic, or other person involved in performing hemostasis on a patient 90. In some contexts, the provider 92 could be a family member, friend, or other type of non-professional provider of services or even the patient 90.

The range of potential providers 92 who may find the apparatus 100 desirable is commensurate with the broad range of contexts that the apparatus 100 can be used. For example, in the context of treating animals, the provider 92 could be a veterinarian or veterinarian's assistant. In the context of human patients 90, the apparatus 100 can be used in the context of a variety of different treatment protocols and a variety of different medical conditions.

For example, the apparatus 100 was originally conceived for the purpose of serving providers 92 involved in radial catheterizations. However, just as the apparatus 100 can be utilized in a broader range of contexts, the apparatus 100 can also be utilized with respect to a broader range of providers 92.

C. Sensor Apparatus

The hemostasis sensor apparatus 100 is an apparatus 100 that is used to capture information about the blood flow of a patient (a blood flow attribute 87) and share or otherwise make available that information (a sensor reading 88) to a provider.

The apparatus 100 can be used in a variety of different contexts, but is typically used as part of a large set of interactions between the patient 90 and the provider 92. As indicated by the various arrows in FIG. 1a, the apparatus 100 can directly interact with both the patients 90 and providers 92. For example, the apparatus 100 is placed on the patient 90 and can be capable of capturing various sensor metrics from the patient 90, including but not limited to information relevant to hemostasis (the process for stopping bleeding) and patency (the objective of sustaining proper blood flow). Such information can then be made accessible to the providers 92.

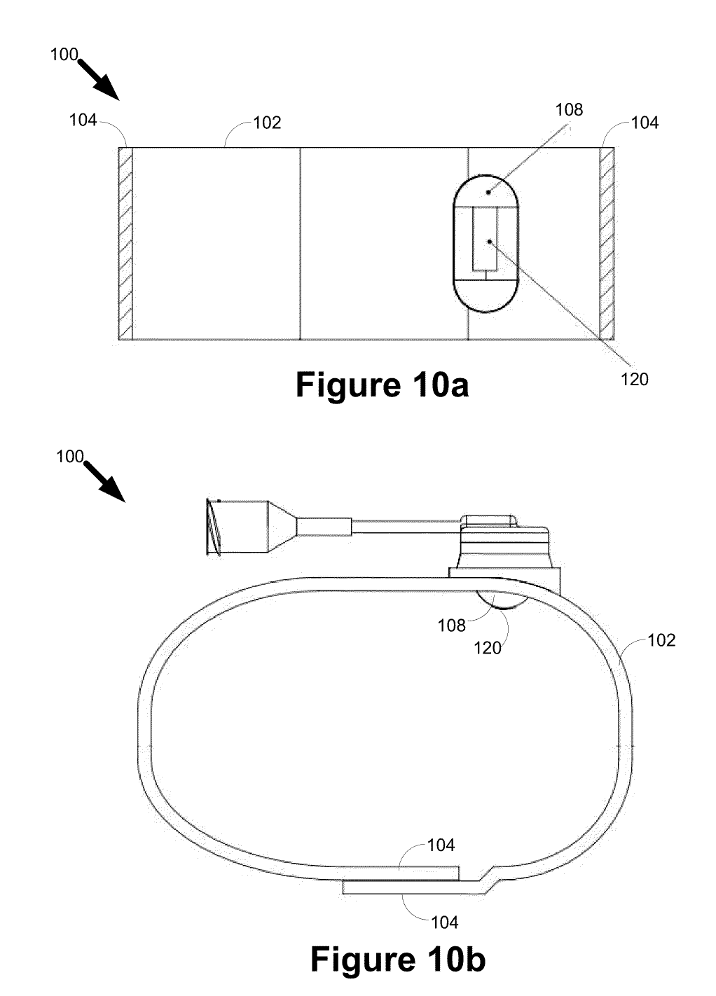

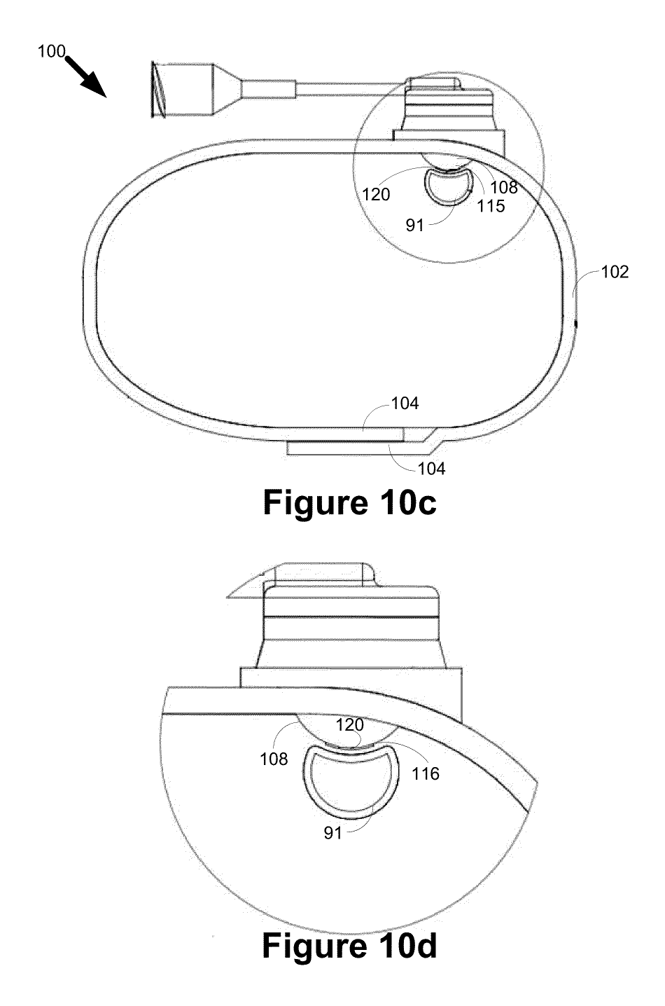

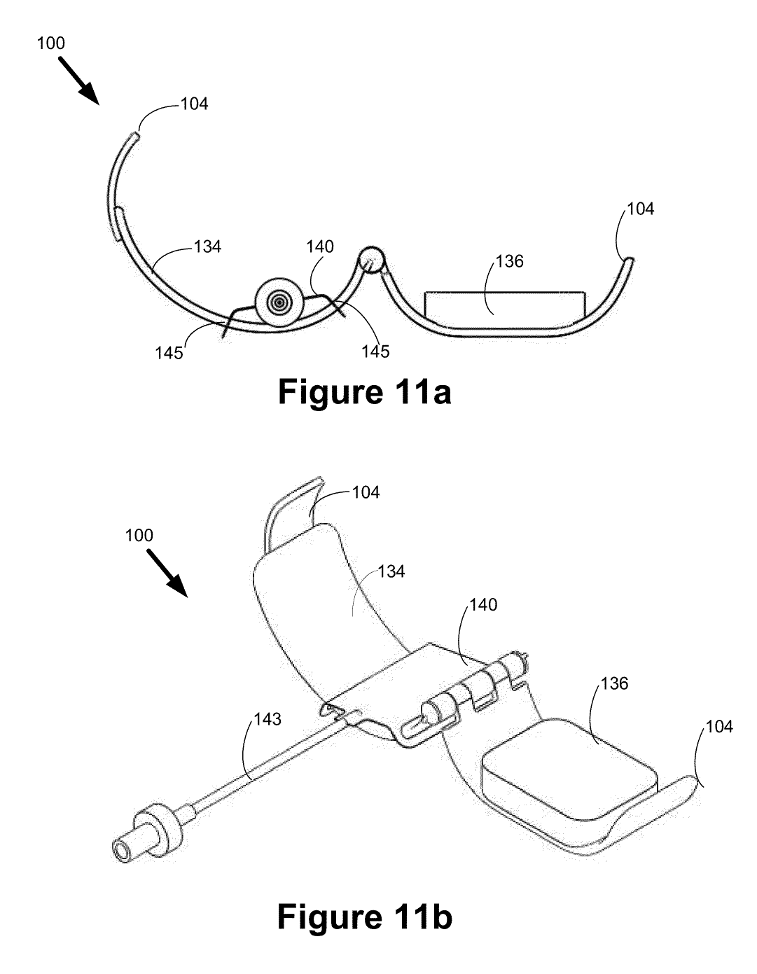

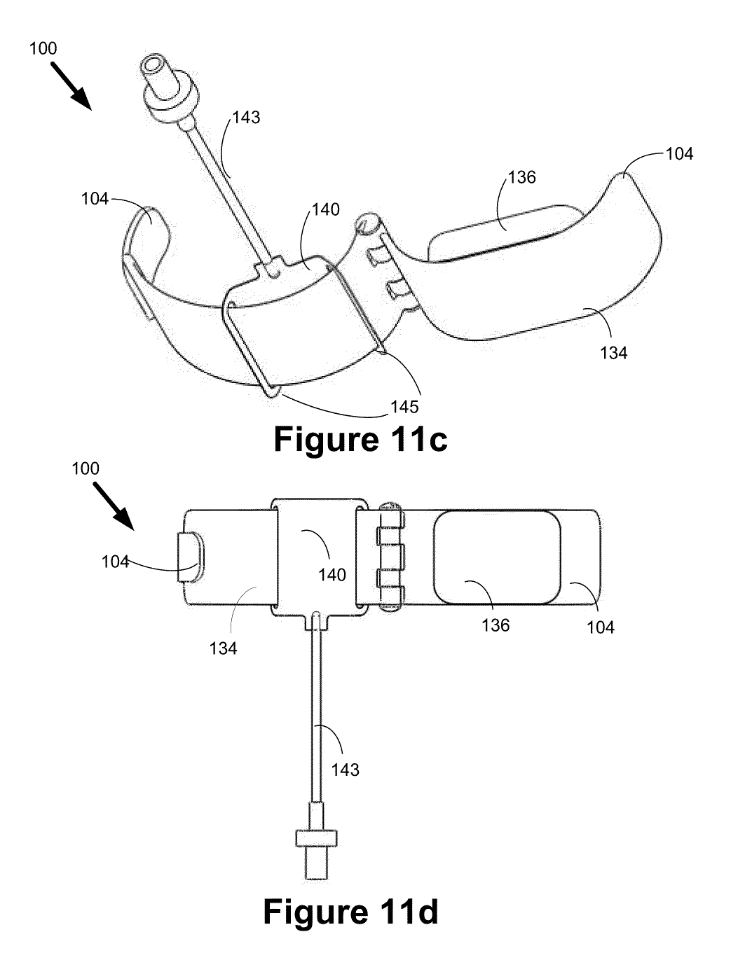

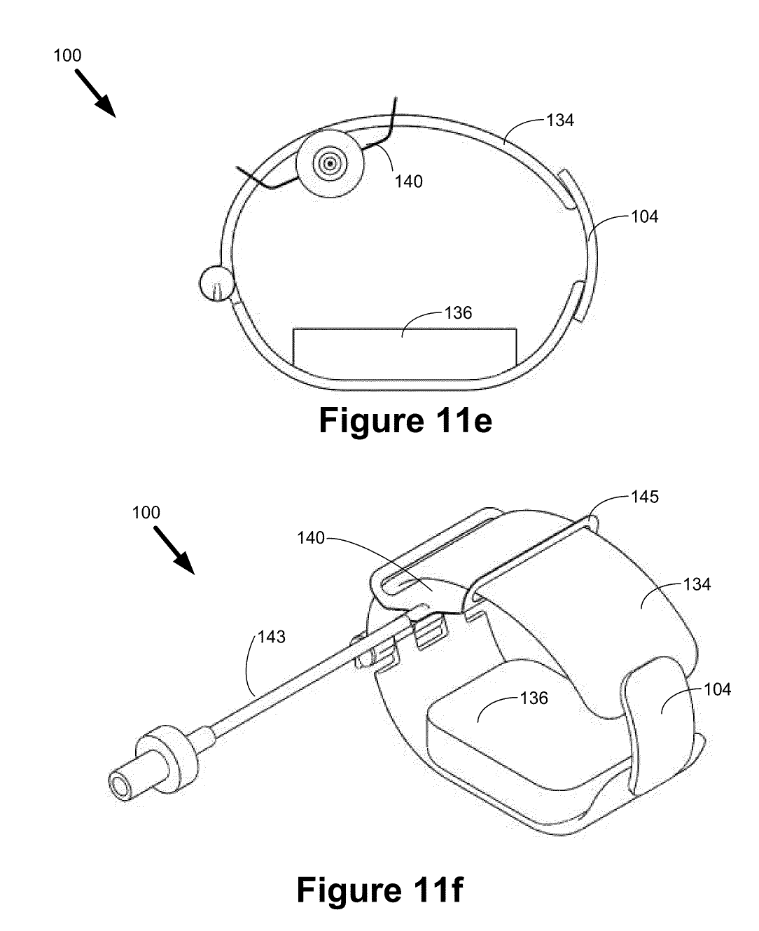

The sensor apparatus 100 can include a wide variety of different components. Some embodiments of the sensor apparatus 100 will involve fully integrated hemostasis devices designed to both stop patient bleeding as well as monitor the patient's 90 pulse at or near the puncture site 89. See FIGS. 10a-11f. The sensor apparatus 100 can also be implemented in a fully stand-alone manner, totally separate and independent of the equipment used to stem the bleeding from the puncture site 89. Many embodiments of the sensor apparatus will involve some level of integration with the hemostasis device that is between full integration and full independence.

As illustrated in FIG. 1a, two components of the sensor apparatus 100 are the sensor component 110 for capturing one or more blood flow attributes 87 from the patient 90 and a communication component 111 for making that information accessible to the provider 92 in the form of a sensor reading 88. As discussed both above and below, the sensor apparatus 100 can be implemented as a highly integrated hemostasis device or it can operate as a stand-alone device used during the hemostasis process.

1. Sensor Component

A sensor component 110 is potentially any mechanism or structure that can be used to capture information relating to the puncture site 89 of the patient 90 or the area around the puncture site 90 of the patient. Sensor components 110 can utilize electronic means, mechanical means, optical means, combinations thereof, and/or other sensors technologies known in the prior art.

In many instances, the desired patient attribute 87 being sensed relates to the pulse or blood flow of the patient 90. However, different embodiments of the sensor 100 can include additional types of information that may be helpful or relevant to a particular patient 90 in a particular setting or context. A single embodiment of the sensor apparatus 100 can include multiple different types of sensor components 110. A sensor component 110 can also be implemented and used as a stand-alone device outside the context of any hemostasis device, as a separate component used in conjunction with one or more other hemostasis devices, or as part of a fully integrated hemostasis device.

The sensor apparatus 100 can include a wide range of different categories and subcategories of sensor components 110. Sensor components 110 can be differentiated on the basis of the underlying technology used to capture sensor readings 88.

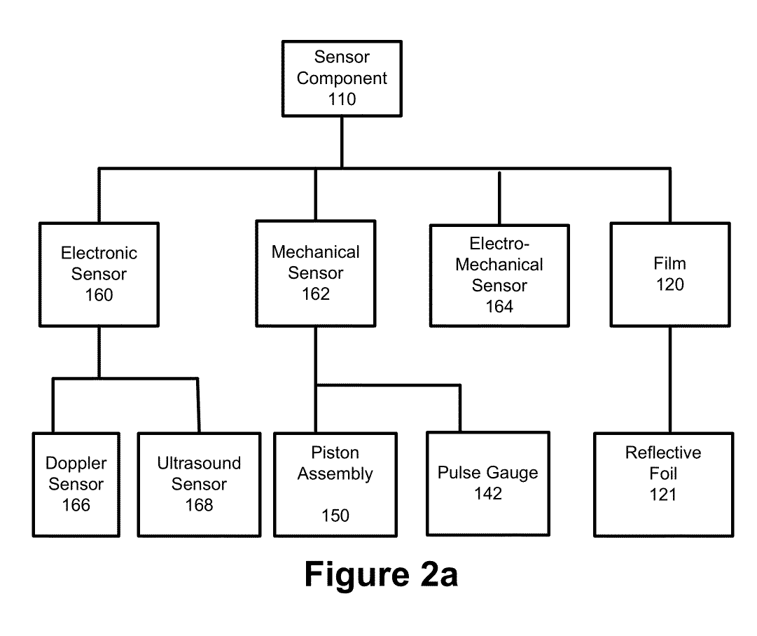

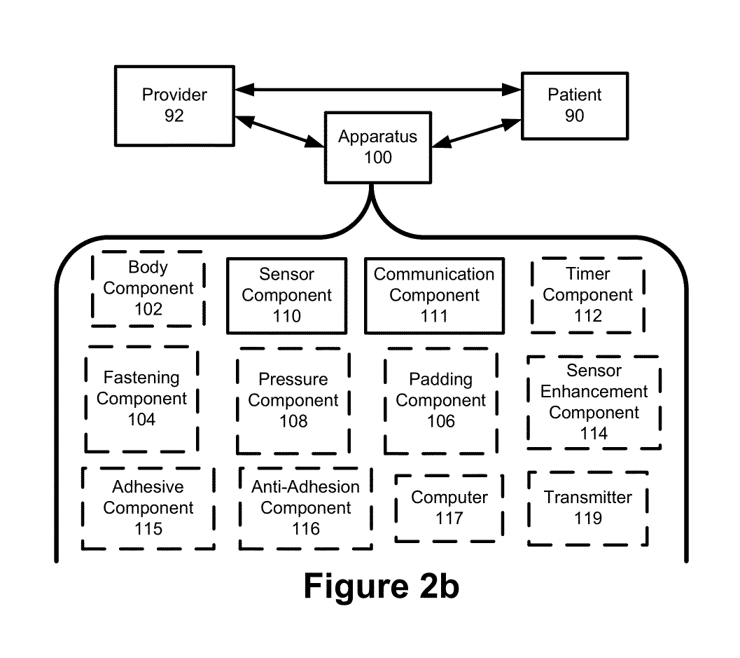

FIG. 2a is a hierarchy diagram illustrating an example of different categories and subcategories of sensor components 100, such as an electronic sensor 160 (including but not limited to a Doppler sensor 166 and an ultrasound sensor 168), a mechanical sensor 162 (including but not limited to a piston assembly 150 and a pulse gauge 142), an electro-mechanical sensor 164, and a film (including but not limited to a reflective foil 121).

a. Electronic Sensors

One category of sensor components 110 is the category of electronic sensors 160. An electronics sensor 160 is a sensor component 110 whose internal operation is provided by electronic subcomponents within the sensor component 110.

Examples of electronic sensors 110 that can be incorporated into the apparatus 100 include: pressure sensors; force sensors; feedback sensors (visual, audio, and/or both); strain sensors; strain gauges; movement sensors; gyroscopes; fiber optic sensors for measuring pressure, force, strain and/or motion; printed ink sensors; piezo electric sensors; and/or potentially any other electronic sensor known in the art.

Included within the category of electronic sensors 160 are sensors whose internal electronics are used to capture sensor readings 88 based on sound, such as a Doppler sensor 166 (which captures sensor readings 88 based on the physics principle that sound pitch increases as the source of the sound moves toward the listener and decreases as is the source moves away) and an ultrasound sensor 168 (which captures a sensor reading 88 based on the physics principle that sound reflected back from an aimed target can be detected back at its origin).

Doppler and Ultrasound technologies can provide information about blow flow attributes or images of the patient's vasculature below the surface of the skin.

There are numerous types of electronic-based sensor technologies not illustrated on FIG. 2a that are known in the prior art outside the context of hemostasis that can be viable options for electronic sensors 160 in a hemostasis sensor apparatus 100 intended for use during the hemostasis process of a patient 90.

b. Mechanical Sensors

A mechanical sensor 162 is a category of sensor component 110 defined by the use of mechanical mechanisms to capture sensor readings 88 relating to the patient 90.

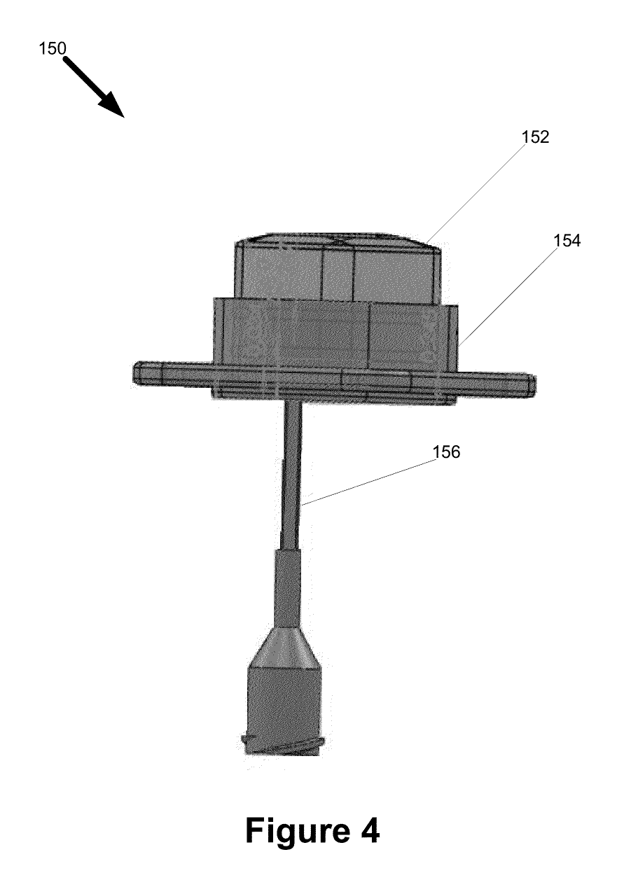

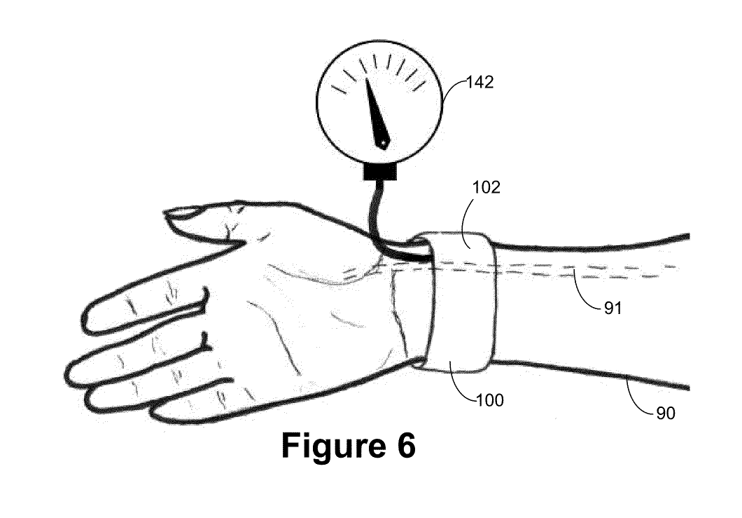

Examples of mechanical sensors 110 that can be incorporated into the hemostasis sensor apparatus 100 include: a piston assembly 150 (see FIG. 4); a pulse gauge 142 (see FIG. 6); a pressure gauge; a manometer gauge; and other mechanical and/or micro-mechanical sensor known in the prior art outside the context of the hemostasis process.

The primary purpose of the sensor apparatus 100 is to capture information about the blood flow of the patient 90 (i.e. one or more blood flow attributes 87). The pulse of blood through the blood vessels of the patient 90 is ultimately a mechanics-based process, and thus there are a wide number of options for use of mechanical sensors 162 that are known outside the hemostasis context that can be utilized as mechanical sensors 162 with respect to the apparatus 100.

1. Piston Assembly

FIG. 4 is a diagram illustrating an example of a piston assembly 150. As illustrated in the Figure, a floating piston 152 is able to move vertically within an oval cylinder 154. An inflation tube 156 provides for the introduction of air/air pressure into the assembly 150.

2. Pulse Gauge

FIG. 6 is a diagram illustrating an example of a sensor apparatus 100 that includes a pulse gauge 142 as a mechanical sensor 162. The pulse gauge 142 is also an example of a sensor component 110 that is integrated with a communication component 111 in that the pulse gauge 142 includes a display to communicate the sensor reading 88 to the provider 90 in a way that the piston assembly 150 does not.

c. Electro-Mechanical Sensors

Returning to FIG. 2a, the sensor apparatus 100 can also utilize an electro-mechanical sensor 164 as a sensor component 110. Electro-mechanical sensors 164 are hybrid sensors that combine certain aspects of both electronic sensors 160 and mechanical sensors 164.

d. Film and other Visual Pulse Indicators

For many if not most of the sensor components 110 discussed above, the sensor component 110 can be coupled with a wide variety of different communication components 111. There are however some sensor components 110 which do not need to be coupled with a communication component 111 because the sensor component 110 is itself the means of communicating the sensor reading 88 to the provider 90. A film 120 is an example of a sensor component 110 that also inherently and inseparably performs the function of both a sensor component 110 and a communication component 111. Films 120, including but not limited to reflective foils 121, can also be referred to as “visual pulse indicators”.

Film 120 can function as a sensor component 110 for pulse/blood flow of the patient 90 because film 120 is flexible, and the position/shape of film 120 can fluctuate with the pulsing of the blood at the location under the film 120. Film 120, such as a reflective foil 121, can readily convey information to providers 92 without the use of electricity or moving parts.

There are a wide variety of different reflective materials that can be used to comprise the reflective foil 120, including but not limited to polyester. The foil 120 moves with pulsing blood up and down, changing color with each cycle (typically from red to yellow, although different color configurations are possible). When too much pressure is applied to the puncture site 89, blood flow is constricted and visual pulsation is not seen. When too little pressure is applied to the puncture site, a relatively small portion of the reflective foil 120 displays any type of color and bleeding may occur.

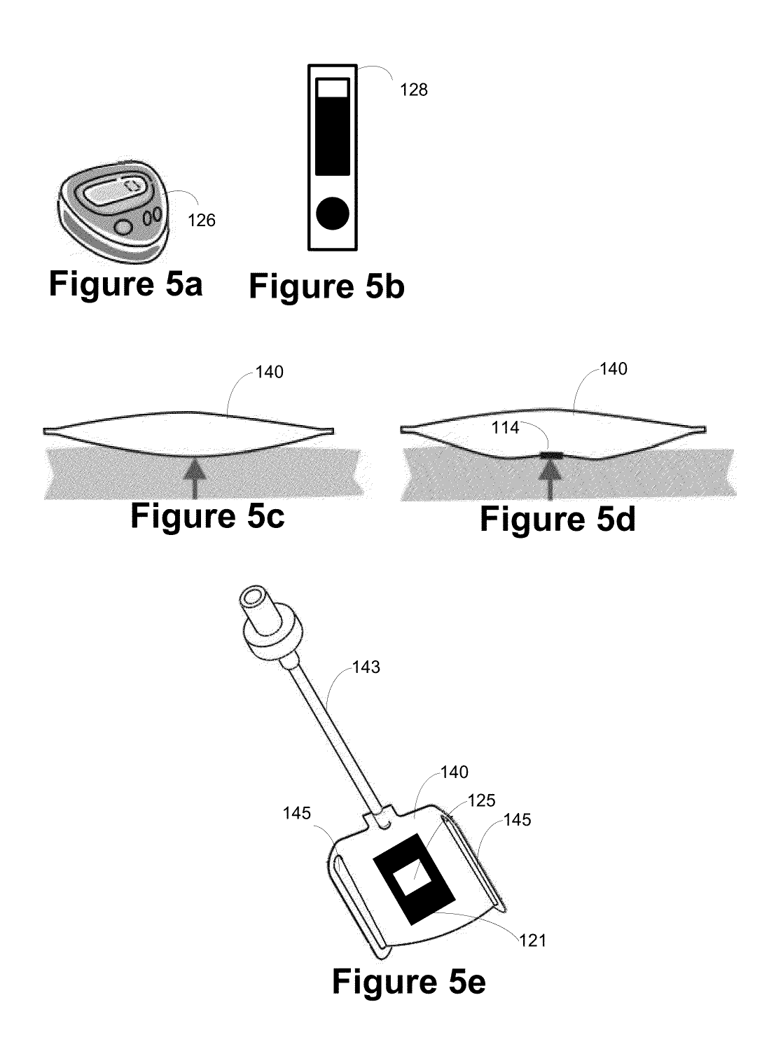

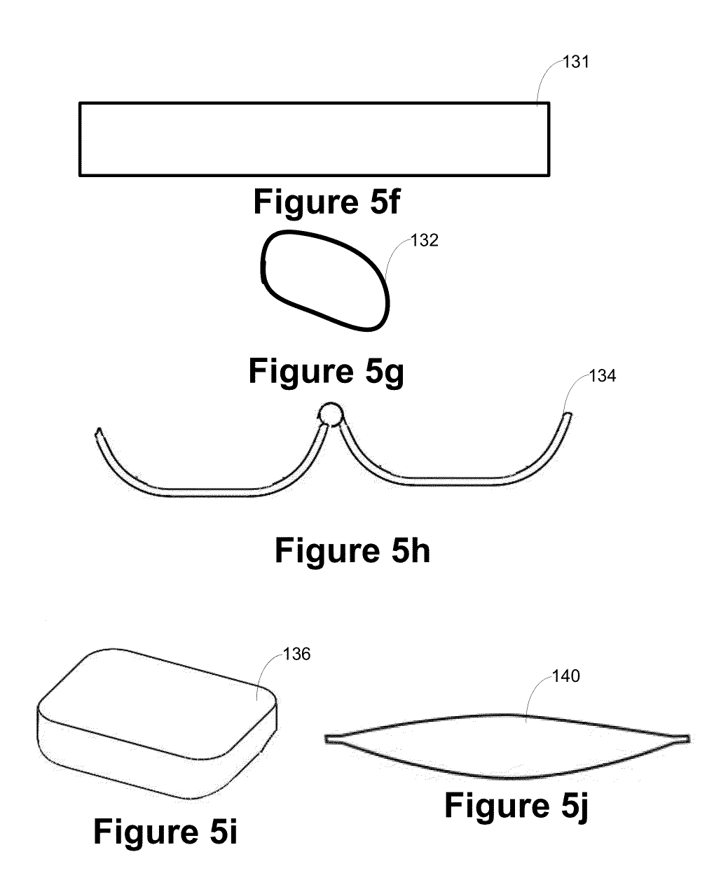

The use of film 120 such as a reflective foil 121 can also be readily augmented and enhanced by a sensor enhancement component 114 (see FIG. 2b) such as: light sources to aid visualization (fluorescent, incandescent, LED, or other); magnifier/magnifier lens; liquid filled balloon to aid visualization; and other types of sensor enhancement components 114 (see FIGS. 2b and 5d).

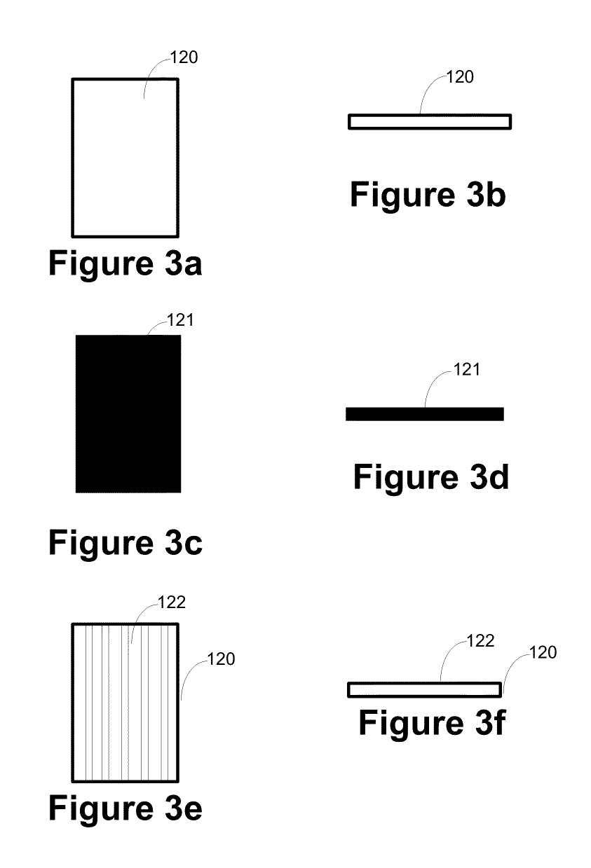

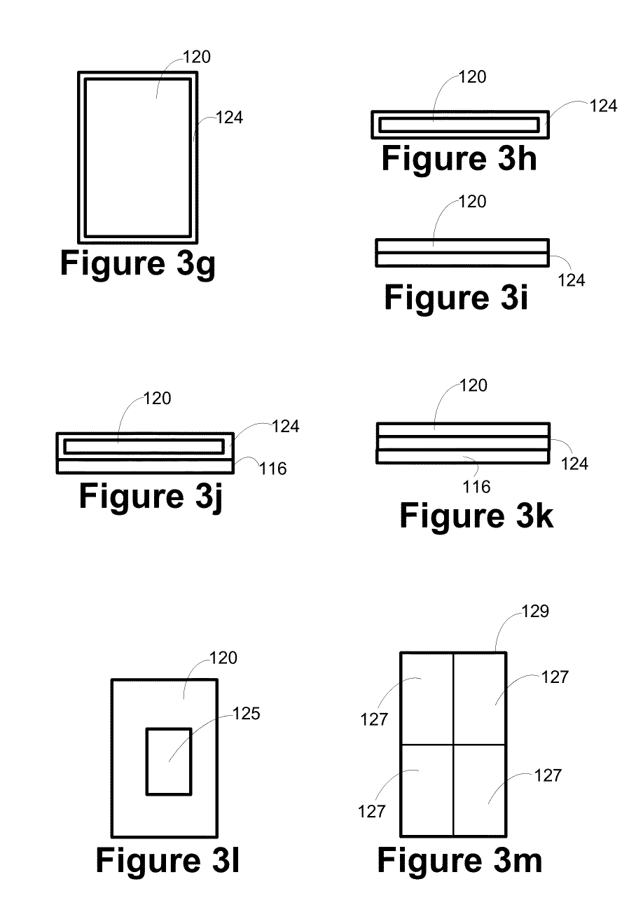

FIG. 3a is a diagram illustrating an example of a top-view of a film 120 as a sensor component 110. FIG. 3b is a diagram illustrating an example of a side-view of the film 120 illustrated in FIG. 3a.

1. Reflective Foil

FIG. 3c is a diagram illustrating an example of a top-view of a reflective foil 121 as a sensor component 110. FIG. 3d is a diagram illustrating an example of a side-view of the foil 121 illustrated in FIG. 3a. Reflective foil 121 can be a very effective type of film 120 sensor component because reflective foil 121 maximizes the use of the existing ambient light.

A substrate of reflective foil 121 receives input from pulsation of the radial artery 91. The pulsation can cause movement of the foil 121. Ambient light shining on the foil 121 will be reflected at a different angle due to movement caused by pulsation. The foil 121 will have a different or changing appearance due to light reflecting at different angles.

One embodiment of a reflective foil 121 is a hologram foil 121. The appearance or color of the hologram will be different or change as light reflects from it at different angles. A pattern 122 of the hologram may facilitate or optimize the performance or capabilities of the foil 121, such as facilitate or optimize the ability to visually see the foil 121. The pattern 122 may be plain, repeating, random, an image or images, a word or words, a geometric shape or a combination of these patterns.

2. Patterns

FIG. 3e is diagram illustrating an example of a top-view of a film 120 with a pattern 122 such as a Moiré line pattern. A wide variety of different patterns 122 (including but not limited to Moiré line patterns) can be implemented onto film 120 such as reflective foil 121. FIG. 3f is a diagram illustrating an example of a side-view of the sensor component 110 in FIG. 3e. Patterns 122 can be very useful in the context of film 120 because patterns 122 enhance the ability of film 120 to provide visual impressions that distinguish between acceptable patient pulse conditions, and a pulse indication of constricted blood flow.

Moiré patterns can be a particularly desirable type of pattern 122 to use on a reflective foil 121 being used to capture blood flow attributes 87. Moiré patterns are two images capable of moving with respect to each other, forming in the aggregate a different image than the two composite images. For example, two lines could combine to form a single line in response to the vibrations generated by the pulse at the puncture site 89. A wide variety of different patterns 122 could be used in conjunction with the film 120.

3. Sheath

FIG. 3g is a diagram illustrating an example of a top-view of a film 120 (an example of a sensor component) encased in a sheath 124. FIG. 3h is a diagram illustrating an example of a side-view of a film 120 (an example of a sensor component) encased in a sheath 124. FIG. 3i is a diagram illustrating an example of an alternate side-view of a film 120 (an example of a sensor component) encased in sheath 124.

A sheath 124 can serve a variety of purposes with respect to a sensor component 110 such as film 120. In addition to shielding the film 120 from direct contact with the patient 90 or other apparatus components, the sheath 124 can also provide an additional layer of material to impact the visual impression of the film 120. In many embodiments, the sheath 124 will be transparent or substantially transparent. A sheath 124 can also be associated with a pattern 122 that is distinct from the pattern 122 on the film 120, but that interacts with the pattern 122 on the film 120.

4. Adhesion and Anti-Adhesion Components

FIG. 3j is a diagram illustrating an example of a side-view of a film 120 (an example of a sensor component) encased in a sheath 124 with an anti-adhesion layer 116 positioned between the sheath 124 and the patient 90. FIG. 3k is a diagram showing the view of FIG. 3j turned 90 degrees. Film 120 can benefit from the use of an anti-adhesion component 116 (or alternatively an adhesion component 115) in a variety of different circumstances and contexts. Film 120 unlike many other types or subtypes of sensor components 120 may require an adhesion component 115 or an anti-adhesion component 116 because film 120 requires direct or indirect physical contact with the skin surface at or near the puncture site 89.

5. Window

FIG. 3l is a diagram illustrating an example of a top view of a film 120 that includes a window 125 for viewing the puncture site 89. A window 125 provides providers 92 with an opportunity to directly view the puncture site 89 even if a film 120 such as reflective foil 121 is placed over the puncture site 89 for the purposes of sensing the pulse 87 of the patient 90.

6. Grids and Zones

FIG. 3m is a diagram illustrating an example of a top view of a film 120 that is comprised of a grid 129 that includes 4 zones 127. By dividing up a foil 121 or other film into multi-dimensional grids 129 of multiple zones 127, the sensitivity of the film 120 to different ranges of pulse information is enhanced because different zones 127 within the film 120 can provide separate and distinct sensor readings 88 to the provider 92.

Although the grid 129 disclosed in FIG. 3m is a 2×2 grid, other grid configurations are possible. For example a 3×3 grid may be particularly desirable.

The sensor readings 88 produced by the sensor component 110 can vary from patient 90 to patient 90 due to a variety of conditions. There may be the need to quantify the output 88 from the sensor apparatus 100. A grid 129 of zones 127 approach provides one way for film 120 to generate a quantitative sensor reading 88. The pattern may be a 3×3 grid pattern. The output signal may be visually seen in all nine zones of the 3×3 grid if it is strong. If the signal is weak, it may only be seen in one or two zones 127 of the grid 129. The signal may be seen only in all three areas of a row or a column of the grid 129. The number of zones 127 of the grid 129 that exhibit the output signal serves to quantify the signal. The grid 129 may have different patterns other than a 3×3 grid pattern in order to quantify the output signal.

2. Communication Component

A communication component 111 is the mechanism that makes a sensor reading 88 accessible to a provider 90. As discussed above, in the context of a film 120 as the sensor component 110, the film 120 can also serve as the communication component 111. In the contexts of other types of sensor components 110, the communication component 111 will often be permanently fixed to the sensor component 110. In other contexts (particularly as modularized electronics increasingly have connectivity capabilities), the communication component 111 can be readily removed and connected to different sensor components 110.

Examples of communication components 111 include but are not limited to all types of computer displays 144 (see FIG. 8), gauges 142 (see FIG. 6), audio speakers, film 120, connectivity technologies, and other technologies known to the art outside the context of communicating sensor readings 88 as part of a hemostasis process.

One purpose of the communication component 111 is to provide providers 92 with an alert if the pulse of the patient 90 is unduly impeded by the application of pressure to the puncture site 89. An alert can be communicated through one or more human senses, typically sound and/or sight. Electronic communication components 111 can be used in conjunction with communication networks to disseminate information to individuals not physically present with the patient 90. In some embodiments, alert conditions can be defined on criteria that are specific to the patient 90 so that alerts are more freely triggered for particularly vulnerable patients 90.

3. Optional Components

FIG. 2b is a block diagram illustrating an example of an apparatus 100 that includes a sensor component 110, a communications component 111, and a variety of optional components that can be included in a hemostasis sensor apparatus 100, with such optional components including a body component 102, a timer component 112, a fastening component 104, a pressure component 108, a padding component 106, a sensor enhancement component 114, an adhesive component 115, and anti-adhesion component 116, a computer 117, and a transmitter 119.

a. Body Component

A body component 102 is the structure or mechanism used to keep the other components of the apparatus 100 together.

In many embodiments, the body component 102 is a strip 131 (see FIG. 5g), a flexible band 130, a semi-flexible/semi rigid band 134 (see FIG. 5i), an elastic loop 132 (FIG. 5h), a rigid substrate with predefined folds and/or hinges, or virtually any other geometry or configuration that can provide a suitable platform for connecting/hosting components of the apparatus 100.

For example, in the context of radial catheterization, the body component 102 can be a strap or band that either fully or partially wraps around the arm of the patient 90.

The body component 102 can be made a variety of different materials. For example, the body component 102 can be fully flexible, semi-rigid, or even fully rigid. In some instances it can be desirable for the body component 102 to be transparent to facilitate the functionality of a sensor component 110 or for some other purpose, while in other instances the body component 102 can be translucent or even opaque.

In some embodiments, the body component 102 is the same component as a fastening component 104. For example, an elastic band can serve as both the body component 102 and the fastening component 104.

b. Fastening Component

A fastening component 104 is potentially any mechanism or structure that can secure the apparatus 100 to the applicable location on the patient 90. Examples of fastening components 104 include buckles, snap-hooks, buttons, zippers, adjuster bars, slides, cord locks, zipper pulls, modular buckles, hook-and-loop fasteners, continuous elastic loops, continuous inflatable loops, fabric fastening tape comprised of a dense arrangement of tiny nylon hooks and an interlocking nylon pile (i.e. ®VELCRO), or any other example of a fastening technology or apparatus.

In some embodiments, the fastening component 104 is inherent in the nature of the body component 102. For example, no separate fastening component 104 is required if the body component 102 is an elastic loop 132 or an inflatable loop.

c. Padding Component

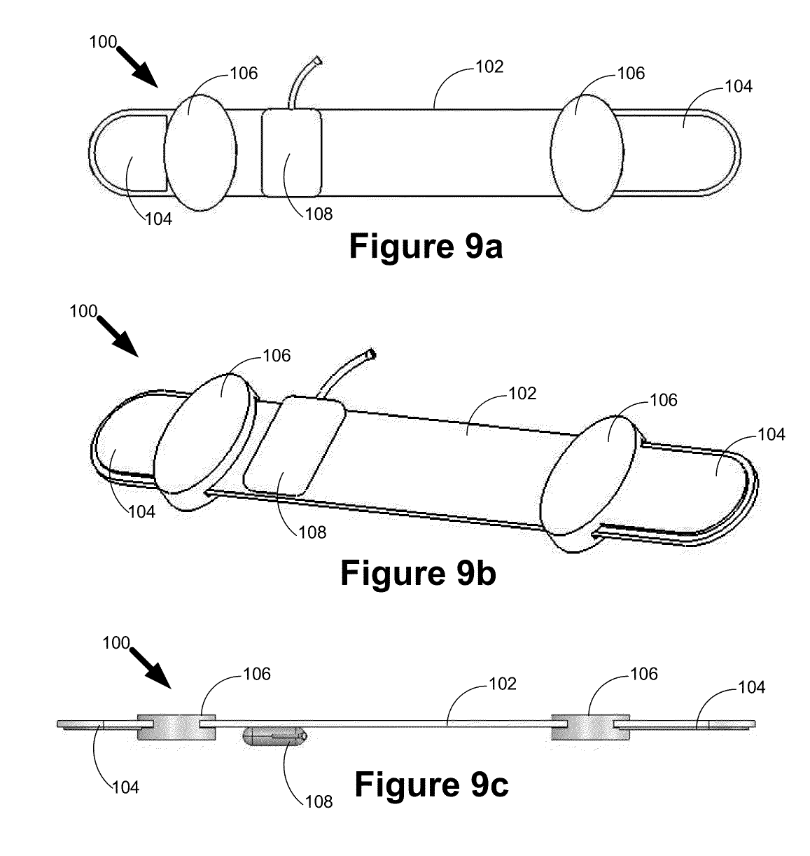

A padding component 106 is potentially any mechanism or structure that can make the apparatus 100 more comfortable for the patient 90. Each embodiment of the apparatus 100 will typically involve one or more padding components 106, such as adjustable pads (see FIGS. 9a-9c), foam pads 136 (see FIG. 5j), or inflatable pads (i.e. balloons 140). In some instances, the padding component 106 is not separate and distinct from the body component 102. For example, in the context of an inflatable band serving as the body component 102, the body component 102 itself can inherently possess the desired padding attributes. By way of further example, balloons 140 discussed below can constitute padding components 106, pressure components 108, and sensor enhancement components 114.

d. Pressure Component

A pressure component 108 is potentially any mechanism or structure that can apply pressure to the puncture site 89 of the patient 90. In many embodiments of the apparatus 100, the pressure component is one or more balloons 140, such as a pneumatic balloon inflated with a gas such as air, a hydraulic balloon inflated with a liquid such as water or saline, an adjustable balloon of any type, a balloon shaped to guide the direction of force or pressure, or some other type of flexible material.

Examples of non-balloon based pressure components 108 can utilize mechanical expansion rather than inflation of a balloon. In many embodiments of the apparatus 100, the pressure component 108 is configured to gradually release pressure over a particular period of time. For example, in the context of radial catheterization, it may be desirable for pressure to automatically reduce over a period of time that is typically between 45-150 minutes.

In some embodiments, the pressure component 108 is not necessarily separate and distinct from the body component 102. For example, in the context of an inflatable band serving as the body component 102, the body 102 can inherently possess the desired attributes of the pressure component 108.

Balloons can be particularly useful in embodiments of the sensor apparatus 100 that involve integrated hemostasis bands because balloons serve so many useful potential purposes, i.e. patient comfort, pressure adjustment, and sensor enhancement. It can thus be useful to use multiple balloons in the context of a single hemostasis apparatus 100.

e. Balloons

A balloon 140 can perform a wide variety of different functions with respect to the sensor apparatus 100. A balloon 140 can simultaneously function as a padding component 106, a pressure component 108, and/or a sensor enhancement component 114. Balloons 140 are also capable of being adjusted through a process of inflation/deflation, and thus the use of balloons 140 can enhance the function of the apparatus 100 at the same time that the comfort of the patient 90 is improved.

Balloons 140 can be used to enhance the sensitivity of the sensor component 110. The flexibility of a balloon 140 (see FIGS. 5c, 5d, and 5e) can make a balloon a useful sensor enhancement component 114 whether the corresponding sensor component 110 is a film 120 such as reflective foil 121 or some other type of sensor component 110. The puncture site 89 of the patient 90 is typically quite small and flat, and a balloon 140 can be an effective amplifier for blood flow attributes 87 sensed by the applicable sensor component 110. As illustrated in FIG. 5d, the effectiveness of a balloon 140 in performing the function of a sensor enhancement component 114 can be further enhanced by other sensor enhancement components 114, such as a “middle member” or “air pocket” as displayed in FIG. 5d. As illustrated in FIG. 5e and in FIGS. 10a-10d, a balloon 140 can perform the function of a pressure component 108 while in close proximity to the sensor component 110 because in many embodiments the balloon 140 can function as a sensor enhancement component 114.

The hemostasis sensor apparatus 100 may be designed in a variety of different ways and may contain a variety of different components. Some embodiments of the hemostasis apparatus 100 can contain two balloons 140. In some embodiments, each balloon could serve simultaneously as a padding component 106, a pressure component 108, and/or a sensor enhancement component 114. In other embodiments, the different balloons 140 could have more specialized functions

One balloon 140 would serve as a pressure component 108. This balloon 140 would be positioned over the puncture site 89 and inflation of the balloon 140 would apply pressure to stop bleeding, which is hemostasis. A second balloon 140 would serve as a sensor enhancement component 114. The second balloon could be adjacent to the first balloon, or positioned in a proximity that would facilitate or optimize its performance or capabilities. One possible position for the sensor enhancement balloon is distal or downstream to the first balloon serving as a pressure component 108. The second balloon could be attached to the film 120 or some other sensor component 110. The design of the hemostasis apparatus can be that both balloons are inflated at the same time. Alternately, the design could be that the balloons are inflated separately. The hemostasis apparatus can be designed that both balloons are integrated into the same body component, or the balloons could be contained in separate body components. The two balloons may have a fixed position relative to each other, or their position relative to each other can be adjusted as needed. Both balloons could be inflated with the same fluid, such as pneumatically with a gas like air or hydraulically with a liquid like water. Alternately, the balloons could be filled with different fluids. The balloons may be inflated to the same pressure or to different pressures. The balloons may have the same geometric shape or different shapes. The balloon volumes, inflated or deflated, may be the same or different. The balloons may be made from the same materials or different materials. The balloons may be made from the same materials, but different amounts of the same material. For example, both balloons are made from the same material, but one balloon is made from thinner material than the other. The balloons could be put on the patient at the same time during the medical procedure or at different times.

f. Timer Components

Returning to FIG. 2a, a variety of timer components 112 can be incorporated into the apparatus 100 to accomplish a variety of purposes. A timer component 112 can be used to time the hemostasis process itself. A stopwatch 126 (see FIG. 5a) or ink release strip (see FIG. 5b) can be incorporated into the apparatus 100. If the apparatus 100 provides for communicating with exterior systems 113, the exterior system 113 can provide this information. Timer components 112 can also be incorporated into the processing of the sensor components 110, determining how often sensor readings 88 are captured and/or transmitted.

g. Sensor Enhancement Component

A sensor enhancement component 114 is any device or structure that can enhance the ability of a sensor component 110 to capture meaningful sensor readings 88. The variety of different sensor enhancement components 114 is commensurate with the variety of different types of sensor components 110. For example a variety of enhancement components 114 in a film 120 or reflective foil 121 embodiment will involve lighting at the location of the sensor 110. Balloons 140 which serve to distribute pulse motions from a small largely two dimensional surface into a larger three dimensional surface can be useful sensor enhancement components 114 for a variety of different types of sensor components 110.

h. Anti-Adhesion Component

In some instances, it can be desirable for a surface of the apparatus 100 to be coated with an anti-adhesion component 116. This would be valuable so that when the apparatus 100 is removed from the patient's wrist, it does not damage the puncture site 89 that has just healed. To use a layman's expression, it would not ‘pull the scab off’ the wound. This is particularly likely in the context of a surface that comes into contact with the skin of the patient 90. Medical grade silicone coating is a common example of an anti-adhesion component 116.

i. Adhesive Component

A variety of different adhesive components 115 can be used to: (1) connect different components of the apparatus 100 together; and/or (2) temporarily secure one or more surfaces of the apparatus 100 to the skin of the patient 90. For example, the apparatus 100 can be embodied as a small computer chip that is temporarily secured on the skin of the patient 90. Such an embodiment could benefit from an adhesive component 115 to secure the sensor 110 to the skin of the patient 90 during the hemostasis process.

j. Computer

One or more computers 117 can be incorporated into the apparatus 100. Computers 117 can be particularly useful with embodiments involving electronic sensors 110, electronic communication components 111, and/or exterior systems 113.

K. Transmitter

A transmitter 119, whether wireless or wired, can be used to connect the apparatus 100 to exterior systems 113 or even other components of the apparatus 100.

IV. Continuum from Modular Stand-Alone Embodiments to Fully Integrated Embodiments

As illustrated in FIG. 2a, the sensor apparatus 100 can include a wide variety of different sensor components 110. As illustrated in FIG. 2b, the apparatus 100 can include a wide variety of different components and component combinations since few of the component types are required to be included in the apparatus 100. The implications of FIGS. 2a and 2b when taken together meant that the apparatus 100 can be implemented in vastly different ways.

The sensor apparatus 100 can be implemented as a stand-alone device used to monitor the pulse of a patient 90 at or around the puncture site 89 during a hemostasis process. For example, the apparatus 100 can be implemented as a small electronic chip that includes an electronics sensor 160 and an electronic communication component 111 that is secured to the skin of a patient 90 using an adhesive component 116.

However, in many contexts, it is convenient for providers 92 and patients 90 to integrate the functionality of capturing sensor readings 88 and making sensor readings 88 accessible to providers 92 with functions relating to the hemostasis process itself. Otherwise, multiple devices are used where one would be sufficient, convenient, and potentially more desirable. The sensor apparatus 100 can be embodied in a fully integrated device such that the apparatus 100 itself performs the hemostasis by applying pressure to the puncture site 89.

Between the two polar opposites of full integration and total stand-alone functionality are different degrees of integration. In addition to the variable of degree of integration, there can also be variations in the level of permanence in the integration of components. For example, some embodiments of even a highly integrated apparatus 100 can involve modular components that are capable of being swapped in and out, as desired.

By way of example, the sub-assembly in FIG. 5e is intended to be used in conjunction with the comprehensively integrated apparatus 100 of FIGS. 11a-11f. The sub-assembly in FIG. 5e includes a reflective foil 121 with a window 125 in the center. An inflatable balloon 140 can serve as a pressure component 108, a padding component 106, and a sensor enhancement component 114. An inlet 143 provides for the inflation of the balloon 140.

By way of further example, the various components of the sub-assembly in FIG. 5e are themselves capable of being separated and substituted. Thus, different implementations of the apparatus 100 can differ with respect to a wide variety of different configuration parameters.

V. Apparatus as Part of Hemostasis Process

The apparatus 100 is a medical device used to capture sensor readings 88 that are made accessible to providers 92. The apparatus 100 can also be integrated into the hemostasis process that can used to stop bleeding at the applicable puncture point 89 on the patient 90 and indicate one or more applicable sensor metrics, such as a pulse associated with the patient 90.

The apparatus 100 can be implemented in a variety of different ways and contexts. For example, the apparatus 100 can be used to stop bleeding from the radial artery of a patient 90 after the completion of a catheterization procedure. The catheterization procedure created a needle puncture incision in the patient's wrist and radial artery. The puncture site 89 needs to be closed to stop bleeding. The apparatus can apply the necessary pressure over the puncture site 89 to stop bleeding while the body naturally heals the incision. The stopping of bleeding is hemostasis. The apparatus 100 can also monitor patient attributes, such as indicate a pulse. Indicating a pulse means that blood flow through the lumen of the artery is maintained while the device simultaneously stops bleeding from the artery. Blood flow through the lumen of the artery is important because this means that everything downstream of the incision at the puncture site 89 is still receiving a blood supply.

Many embodiments of the apparatus are embodied as wristbands with the purpose to stop bleeding and indicate the pulse of the patient 90. The apparatus 100 can implement both the function of hemostasis and the function of pulse indication in a single device that is convenient and efficient for use by the provider 92 while being comfortable for the patient 90.

The simultaneous capability guides how the apparatus 100 can be used on a patient 90. It guides by showing if the apparatus 100 is put on too much or too little. If the apparatus 100 is put on too much, it will over compress the artery and there will be no blood flow and thus indicate no pulse. If the apparatus 100 is put on too little there will be bleeding from the artery. There can also be the advantage that the pulse feature is immediate and continuous.

Some embodiments of the apparatus 100 can be integrated into various analytical tools that utilize data to comprehensively manage patient treatment and/or comprehensively monitor patient status.

VI. Catheterization Embodiments

The original inspiration for the apparatus 100 was an integrated wristband embodiment used to stop bleeding from a patient's radial artery. Different alternative embodiments can be applied to radial catheterization contexts, or when dealing with different locations of the body and/or different causes of bleeding. However, the context of hemostasis in the context of catheterization is relevant because catheterization is a planned medical procedure, and it involves a puncture site 89 created by the provider 92, i.e. a puncture site 89 at a predetermined location and a predefined magnitude.

A catheterization procedure can be conducted on a patient 90 through their radial artery. Conducting the procedure through the radial artery is called a transradial approach or transradial access. During the procedure a catheter is inserted into the patient's arterial system through their radial artery. The procedure may use more than one catheter. Catheters will be switched as necessary. Typically, access to the radial artery is near the wrist where the radial artery is close to the surface of the skin. A provider 92 will locate the radial artery by feeling for the patient's pulse. The provider 92 will use a needle to pierce the skin and artery. This will give the provider 92 access to the inside of the artery, which is the lumen of the artery. The catheterization is then conduct through the radial artery. The catheter is removed from the artery when the procedure is complete.

The patient 90 will bleed from their radial artery when the catheter is removed. This bleeding needs to be stopped. Stopping the bleeding is called hemostasis. A typical bandage, like the kind used to stop bleeding from a small cut on a finger, is not capable of stopping the bleeding from the radial artery. It cannot stop bleeding because it cannot overcome the pressure of the blood that is bleeding from the artery. A bandage that can apply enough pressure over the puncture site where the needle pierced the skin is needed. The apparatus 100 in the form of the wristband is a bandage capable of applying the necessary pressure in order to stop the bleeding.

A. Body Component

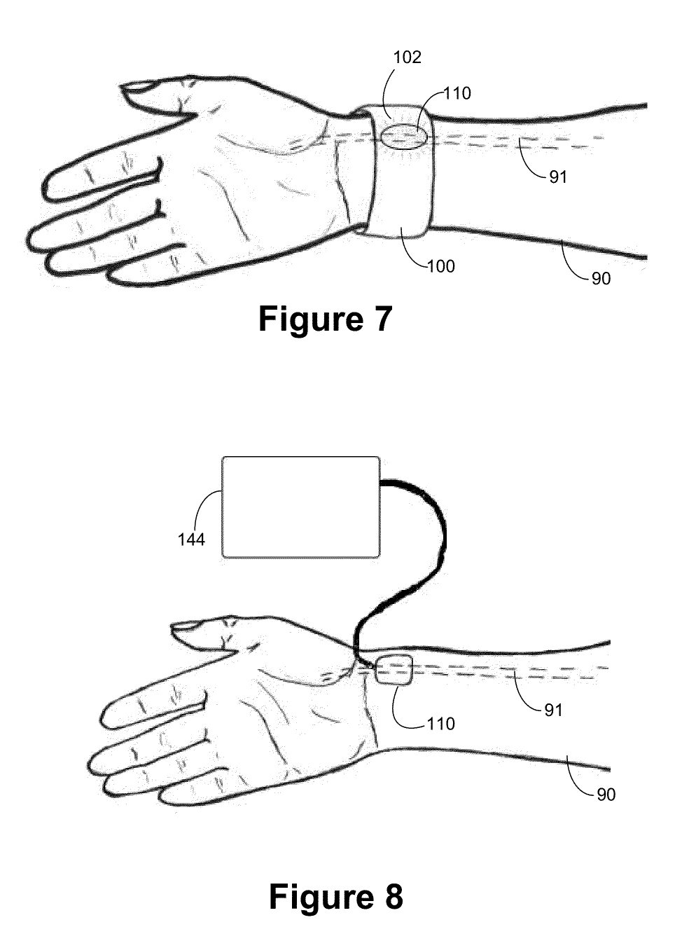

In a wristband embodiment of the apparatus 100, the body component 102 is comprised of a band. Examples of wristband embodiment of the apparatus 100 are illustrated in FIGS. 5g, 5h, 6, 7, 9a-9c, 10a-10d. and 11a-11f. As discussed above with respect to body components 102, a wristband can form an entire loop around the wrist of the patient 90 or the wristband can be limited to a partial arc around the puncture site. In some embodiments of the apparatus 100, the wristband is elastic or even inflatable, allowing the body 102 of the apparatus 100 to also serve as the fastening component 104 and/or the padding component 106.

In some embodiments, the wristband can be transparent or translucent to facilitate visual feedback from the apparatus 100, such as indications from a film 120 or other form of sensor component 110.

B. Fastening Component

As discussed above, a wristband embodiment of the apparatus 100 can include a wide variety of fastening components 104. The fastening component 104 of ®VELCRO straps is illustrated in FIGS. 9a-9c. The wristband embodiment of the apparatus 100 is a medical device used to stop bleeding from the radial artery of the patient 90. The apparatus 100 can be wrapped around the wrist of the patient 90 similar to the way a wrist watch wraps around a person's wrist. The apparatus 100 would have some type of fastening component 104 to keep the apparatus 100 securely fastened around the wrist of the patient 90. There are several ways this can be accomplished. Examples of fastening components 104 can include but are not limited to use of a hook-and-loop fastener such as Velcro®, a buckle like as in a belt, etc. The body 102 of the apparatus 100 may be made as a continuous loop, and the loop can be slipped over the hand onto the wrist. Such a loop can be made from an elastic material. There are several different ways to configure and implement the apparatus 100 so that it stays wrapped around the wrist. It is also envisioned that this feature is easy to use. It would be easy to put on and take off and convenient for both provider 92 and patient 90 alike. Additional examples of fastening components 104 are discussed above.

C. Padding Component

The apparatus 100 can be implemented with one or more features to make it as comfortable as possible for the patient 90 to wear the apparatus 100 while hemostasis of the incision is being achieved. Examples of slidable padding components 106 are disclosed in FIGS. 5j, 5k, 9a-9c, and 11a-11f. It is anticipated that the apparatus 100 would typically be worn between one to two hours after the catheterization procedure. However, the apparatus 100 may need to be worn even longer for some patients 90. Regardless of how long the apparatus 100 is worn, the apparatus 100 may include features to make it more comfortable to wear. One or more padding components 106 may be built into the apparatus 100 to make it comfortable. The padding component(s) 106 may be adjustable in terms of position along the body 102 of the wristband so they can be positioned at desired locations around the circumference of the wrist. The padding component(s) 106 may be made from a variety of materials, such as a foam material or may incorporate an inflated design. There are a variety of ways to make the apparatus 100 as comfortable as possible to wear.

D. Pressure Component

The apparatus 100 can include a pressure component 108 for applying the necessary pressure or force over top of the incision in order to stop bleeding. A variety of ways are envisioned to design this feature. Examples of pressure components 108 are illustrated in FIGS. 9a-11f.

The pressure component 108 may be or may include a balloon. The balloon would be positioned over top of the puncture site. After the apparatus 100 is put on the wrist of the patient 90, the balloon can be inflated to apply the desirable magnitude of pressure. The balloon may be inflated pneumatically with a gas such as air, or hydraulically with a liquid such as water. Other inflation fluids, gases or liquids, may be used if they provide a particular convenience or advantage. The inflation fluid may be particularly advantageous to obtaining hemostasis. The fluid may provide a comfort to the patient with such balloons comprising both the pressure component 108 and the padding component 106.

The fluid used to inflate the balloon may be at a particular temperature to provide convenience and/or advantage. For example, a warm or cool fluid may be advantageous for causing vasodilation or vasoconstriction, which may help obtaining hemostasis. The balloon can be designed so that it can be adjusted and configured as needed. If necessary, the balloon can be inflated more or deflated while the patient 90 is wearing the device.

The balloon may have a variety of shapes. The shape of the balloon may be advantageous to the overall use, design or manufacture of the apparatus 100. For example, the shape of the balloon may help guide the direction of the applied force or pressure. By way of further example, the shape of the balloon may be dictated by how it interfaces with the skin and wrist of the patient 90. In some embodiments, it may be desirable for the balloon to be egg shaped like a toy balloon. In other embodiments, the balloon may be predominantly square or rectangular shape like a pillow or box. The balloon may be irregular shaped if that is advantageous to the use, design or manufacture of the apparatus 100.

The balloon can be made from any of a variety of materials. The materials may be elastic or non-elastic depending on what is needed. In some instances, it may be desirable for the balloon to be comprised of a transparent or translucent material so that the contents of the balloon can be easily viewed.

The apparatus 100 can use mechanical technologies instead of the inflation of one or more balloons to apply force or pressure over the puncture site to stop bleeding and obtain hemostasis. A structure could be built into the apparatus 100 to apply force or pressure. The structure could be balloon-like, but is not inflated. This would be a non-inflated balloon. The non-inflated balloon would be designed to mechanically expand to apply force or pressure. It would mechanically expand instead of inflate.

A cylindrical braid could be used to mechanically expand a non-inflated balloon structure. The ends of the cylinder could be pushed together toward each other causing the cylinder to shorten lengthwise, but also causing it to expand outward in the radial direction. This outward radial expansion could be used to apply pressure or force over the puncture site. This same type of mechanical outward expanding cylindrical braid could be used independent of a non-inflated balloon to apply force or pressure in the context of a film 120 or some other form of sensor component 110.

Other mechanical technologies for implementing the functions of the pressure component 108 can be implemented in the apparatus 100.

E. Sensor Component

The apparatus 100 can include a variety of sensor components 110 to capture useful information about the patient 90 while hemostasis is occurring. One useful embodiment of a sensor component 110 is a pulse indicator feature (the capture of blood flow related attributes 87) that would indicate whether or not blood is still flowing through the radial artery while the apparatus 100 is being worn by the patient 90. It is envisioned that the provider 92 will want to put the apparatus 100 on the patient 90 and apply enough force or pressure to stop bleeding, but not too much force or pressure that the radial artery is compressed and blood cannot flow through the artery. For example, a tourniquet applies enough force and pressure to stop normal blood flow in arteries. The provider 92 would not want the apparatus 100 to create a tourniquet effect. The apparatus 100 would have a feature that indicates if blood is flowing in the radial artery while stopping bleeding from the artery. Various embodiments of sensor components 110 that relate the blood flow through the radial artery can be incorporated into the apparatus 100. Some of those embodiments mimic the process by which a provider 92 manually detects the pulse of a patient 90.

A provider 92 can feel the pulse of a patient 90 by placing a finger over the artery. The provider 92 feels the pulse pushing against their finger. This push is a force, or it can be a pressure. Feeling the cyclic rhythm is how the provider 92 detects pulse. The sensor component 110 may have a feature capable of detecting the pulse and providing real-time feedback to the provider 92. Detecting pulse would indicate to the provider 92 that blood is flowing through the radial artery.

The apparatus 100 can be put on the patient 90, with pressure component 108 applying the necessary force or pressure to stop the radial artery from bleeding. The sensor component 110 would comprise or include a visual indicator for the pulse of the patient 90 to ensure that blood is still flowing in the radial artery. This particular way to use the apparatus 100 obtains hemostasis and maintains blood flow. The feature of the apparatus 100 that monitors pulse would be used to guide how the apparatus 100 is used, how it is put on the patient 90. This would be a unique feature and way to use the apparatus 100, and would be an example of a device in which the benefits and advantages of use are more than the sum of the comprising parts. The apparatus 100 would not merely be put on to stop the bleeding, but would be put on the patient 90 to stop bleeding and maintain blood flow. A feature of such an embodiment of the apparatus 100 would enable the provider 92 to simultaneously apply hemostasis while simultaneously ensuring that blood flow is maintained.

While the patient 90 is wearing the apparatus 100, the provider 92 and other providers 92 can conveniently check both the hemostasis and blood flow as needed or desired. Hemostasis can be checked simply by looking to see if the patient is bleeding from underneath or around the apparatus 100. Blood flow can be checked by looking at the display of the pulse monitor feature. If the apparatus 100 indicates that there is pulse, then the radial artery is not compressed too much. Its lumen is patent and blood is flowing through the artery.

The apparatus 100 can have a pressure component 108 that applies force or pressure over the puncture site in order to stop bleeding. The apparatus 100 can also incorporate an electronic sensor component 110 or some other type of sensor component 110 to monitor the pulse of the patient 90. Electronic sensor components 110 may use one or more sensors that detect force or pressure. Electronic sensor components 110 may have a sensor or sensors capable of monitoring some other parameter of cyclic pulsatile blood flow other than pressure of force. For example, the sensor component 110 may monitor iron in blood. An electromagnetic field may be created around the artery. The flow of iron in blood may change the magnetic field and produce what is known in the prior art as a “Hall Effect”. Detecting a cyclic rhythmic change in the Hall Effect would indicate pulse. Electronic sensor components 110 may have a feature to provide real-time feedback to the provider 92.

Electronics-based sensor components 110 may be configured to monitor a cyclic change in force or pressure in order to detect pulse. If a pulse is detected, the feedback indicates pulse. If no change in force or pressure is detected, the sensor component 110 indicates no pulse. Feedback may be in the form of flashing lights to indicate pulse and no flashing lights to indicate no pulse. Alternately, one color of light may be used to indicate pulse and a different color for no pulse. Other combinations of light, color and flashing are possible for use as feedback. Feedback can also be provided with other visual or auditory means. In some embodiments, feedback can be provided to integrated systems responsible for monitoring and treating patients so that all of the data relating to a patient 90 is easy to access.

If a balloon is used as a pressure component 108, an electronics-based sensor component 110 can be used to monitor the pressure in the balloon. When the artery pulses against the balloon it causes the pressure in the balloon to change. The pressure in the balloon can be monitored by electronics-based sensor component 110 as one way to detect pulse, and thus blood flow in the artery. It may also be possible to have the electronics-based sensor component 110 monitor force on the balloon in order to detect pulse.

The apparatus 100 can include many different types of sensor components 110 designed to capture different metrics relating to the medical status of the patient 90. A single embodiment of the apparatus 100 can include multiple types of different sensor components 110. It is envisioned that more than one type of sensor component 110 may be applicable to monitoring pulse. Sensor components 110 can include strain gauges in the balloon, in addition to the use of force/pressure sensors.

It is envisioned that a radial pulse can apply force or pressure to a balloon serving as a pressure component 108 in the apparatus 100. This force or pressure may result in movement of at least a portion of the balloon surface. One possible design for a pulse monitoring feature is to detect movement of the balloon. Electronics-based sensor components 110 can monitor movement of the balloon surface in order to detect pulse. Electronics-based sensor components 110 may use micro-electro-mechanical systems (MEMS) technology. The MEMS technology may be able to incorporate gyroscope capabilities in order to detect movement. Movement may also be detected using laser technologies.

Some embodiments of sensor components 110 can be based on fiber optic technology to measure pressure, force, strain, motion or movement, temperature, and other potentially relevant metrics, including a pulse.

Some embodiments of sensor components 110 can be based on printed ink technology used to measure force, pressure, and other potentially relevant metrics, including a pulse.

Some embodiments of sensor components 110 can be used on non-electronic mechanical means to monitor the pulse of the patient 90 and other potentially relevant attributes. Pulse can be monitored by detecting force, pressure or movement. It is envisioned that mechanical systems could be used to provide real-time feedback to the provider 92 about pulse. These embodiments could convey information through visual observation or by electronically transmitting the information to other systems responsibility for managing treatment and monitoring patient attributes.

A mechanical pressure gauge could be connected to the inflated balloon. An example of a mechanical pulse gauge 142 is illustrated on FIG. 6. When the radial artery pulses it causes a change in pressure in the balloon. The display of the pressure gauge could be viewed to see if the pressure in the balloon is changing in a cyclic pattern. For example, maybe the needle of the display is oscillating back and forth as the pressure changes.

The apparatus 100 can implemented with a balloon as a sensor component 110 and a provider 92 could simply look at the surface of the balloon and see if it is moving in order to detect pulse. The apparatus 100 could be designed to make it as easy as possible to see movement of the balloon 140. In other embodiments, the balloon 140 functions as a sensor enhancement component 114 and serves to amplify the information conveyed by the communication component 111.

The surface of the balloon 140 that is moving may have a special feature to aid visualization. A hologram or some type of light reflecting or light refracting feature could be put on the balloon to aid visualization. When the surface of the balloon moves because of the pulse, the hologram or light feature would change color because light is hitting it at a different angle. As the balloon surface moves back-and-forth because of the pulse, the color of the hologram or light feature would keep changing. The constant color change would be real-time feedback to the provider 92 indicating pulse and thus blood flow through the artery 91. The apparatus 100 may have a light source to aid visualization. The light source may be of a particular type, source, wavelength, wavelengths or intensity that makes the color change of the hologram or light feature easier to see and thus aids visualization. It is envisioned that fluorescent, incandescent or light emitting diode (LED) may be particularly advantageous to see the color change effect. The light source may have a feature that guides or directs light onto the hologram or light feature. The light source may have a lens that focuses it, or a prism that disperses it. This may be particularly advantageous to seeing the color change effect.

The sensor component 110 of the apparatus 100 can include a magnifying lens or some other form of magnifier to enhance the ability of a provider 92 to view the pulse indicator or any other visually-conveyed patient 90 attribute conveyed by a sensor component 110. A Fresnel lens is one example of a possible lens that can be incorporated into the apparatus 100.

One potential embodiment of the sensor component 110 is a balloon that may be partially or completely inflated with a liquid in order to aid visualization of the balloon surface. When the surface of the balloon moves because of pulse, it disturbs the liquid and the surface of the liquid can be seen to move.

The pulse indicating features described above are envisioned to be able to be incorporated into the apparatus 100 for use as part of an integrated hemostasis device (see FIGS. 10a-10d), but the pulse indicating functionality could also be implemented as a stand-alone device (as is illustrated in FIG. 8). In either instance, the pulse indicating functionality can be implemented using a variety of different types of sensor components 110, including electronics-based sensor components 160, mechanically-based sensor components 162, electro-mechanical sensors 164, and hologram/visualization-based sensor components 121, and/or other forms of sensor components 110.

The pulse indicating feature that can be provided by a reflective foil 121 or some other category of film 120 can be implemented as a stand-alone function, a separate component designed to be used in conjunction with other hemostasis devices, or in the form a fully integrated hemostasis device that provides both the sensor/communication function as well as the hemostasis function. FIGS. 10a-10d illustrate an example of an integrated hemostasis apparatus 100 that includes a reflective foil 121. FIGS. 9a-9c illustrate a hemostasis band to which a wide range of different sensor components 110 and communication components 111 can be temporarily or permanently fixed to. FIG. 8 illustrates an example of a sensor component 110 that is a stand-alone device connected to a display screen 144, an example of a communication component 111. The display screen 144 can also be included in embodiments of the sensor apparatus 100 that are implemented in the form of an integrated hemostasis sensor apparatus 100 that performs hemostasis as well as sensing blood flow attributes 87 during hemostasis. FIG. 6 illustrates an example of an apparatus 100 that includes a pulse gauge 142 that captures a measurement of pulse, such as metric measured in beats-per-minute.

As illustrated in FIGS. 6, 7, and 8, the sensor component 110 is a medical device that will indicate whether or not blood flow is being impeded in the radial artery 91 near the puncture site 89. The sensor component 110 can be placed on a patient's wrist over the radial artery 91. The sensor component 110 can be positioned distal (downstream) to the transradial access site. It can have a low profile so it can be placed on a patient's wrist before the start of a transradial procedure and not interfere with the procedure. The sensor component 110 can provide real-time feedback of pulse indication. The feedback can be audio, visual or some other means. The sensor component 110 only needs to indicate pulse. It does not have to provide a measurement of pulse, such as beats-per-minute. It may provide a measure of pulse such as in FIG. 6, but in many instances, a mere pulse indication is sufficient. In any embodiment, the apparatus 100 and its corresponding sensor component 110 should be sterilized before use.

The sensor component 110 (whether in an integrated apparatus 100 embodiment or as a stand-alone device) can be used to guide placement of a hemostasis band on a patient's wrist after the completion of a transradial catheterization procedure. The hemostasis band is put on the patient's wrist to stop bleeding from the radial artery after the catheterization procedure. A provider 92 would want to put the band on and apply enough pressure to stop bleeding from the artery, but not so much pressure that the artery is compressed too much and blood flow in the artery is stopped. The sensor component 110 indicates whether blood is flowing in the artery by indicating pulse. If the device indicates pulse, then the provider 92 knows that blood is flowing through the radial artery.

It is envisioned that the sensor component 110 (whether in an integrated apparatus 100 embodiment or as a stand-alone device) would be put on the patient 90 before the start of the procedure so the provider 92 knows it is working properly. The catheterization procedure will occlude the radial artery, which means blood will not flow in the radial artery. Because of this, during the procedure the film 120 will indicate no pulse. When the procedure is done and the hemostasis band (either an integrated apparatus 100 or a separate band) is put on correctly without over compressing the artery, the film 120 will indicate pulse. The value of putting the film 120 on before the procedure is to ensure that it is working properly before it is needed during hemostasis. If the film 120 is put on after the procedure and indicates no pulse, then the radial artery is occluded, or the apparatus 100 is not working correctly and it is giving a false-negative. Putting the film 120 on before the start of the procedure eliminates, or at least minimizes the possibility of a false-negative.

Some embodiments of the film 120 involve an electronic film 120 that can integrate with other systems responsible for monitoring the patient 90 and managing the treatment of the patient 90. Embodiments of the film 120 may have a sensor or sensors capable of measuring pressure, force, deflection, movement or some other parameter of cyclic pulsatile blood flow. The sensor component(s) 110 may be integrated computer chips, strain gauges, MEMS, gyroscopes, laser technology, fiber optic technology, etc. as described above.

VI. Integrated Embodiment Illustrations

In the context of integrated apparatus 100 embodiments, there are a wide variety of different ways to implement the sensor apparatus 100. Three potential embodiments are illustrated.

### A. Example 1

### FIGS. 9a Though 9c

FIG. 9a is a diagram illustrating a front view of a hemostasis band 100 that includes a body component 102, a padding component 106, a fastener component 104, a pressure component 108. To that underlying framework, a wide variety of different sensor components 110 and communication components 111 could be added to as an integrated device.

FIG. 9b is a diagram illustrating an example of perspective view of the hemostasis band apparatus 100 illustrated in FIG. 9a.

FIG. 9c is a diagram illustrating an example of a side view of the hemostasis band apparatus 100 illustrated in FIG. 9a.

### B. Example 2

### FIGS. 10a Though 10d

FIG. 10a is a diagram illustrating an example of a front view of an integrated hemostasis sensor apparatus 100 with a film 120 as a sensor component 110, and an inflatable balloon as a pressure component 108. The film 120 is positioned between the pressure component 108 and the radial artery 91 of the patient 90. The apparatus 102 also includes a fastener component 120 in the form of two ®VELCRO strips.

FIG. 10b is a diagram illustrating an example of a side view of the integrated hemostasis sensor apparatus 100 illustrated in FIG. 10a. This illustration shows the two fastener components 104 locked together so that the body component 102 forms a loop.

FIG. 10c is a diagram illustrating an example of a side view of the hemostasis band illustrated in FIG. 10a. In FIG. 10c, an adhesive component 115 is located between the pressure component 108 and the film 120 to securely the position the film 120 on the pressure component 108.

FIG. 10d is a diagram illustrating a close-up detailed view of a portion of FIG. 10c, except that in FIG. 10d an anti-adhesion component 116 is positioned between the film 120 and the artery 91 of the patient 90 so that when the apparatus 100 is removed, the film 120 does not stick to the puncture site 89 and result in “picking the scab”.

### C. Example 3

### FIGS. 11a-11f

FIG. 11a is a diagram illustrating an example of a side view of a sensor apparatus 100 embodied in an integrated hemostasis band. This embodiment of the apparatus 100 utilizes the subassembly illustrated in FIG. 5e that is discussed above. Two fastener components 104 comprised of interlocking clasps can open and close the apparatus 100. The body component 102 is a semi-rigid/semi-flexible band 134 that is substantially transparent. A balloon 140 is the pressure component 108 and the balloon 140 also serves a sensor enhancement component 114. Two gaps 145 on the balloon 140 allow the balloon 140 to slide along the body component 102. An inlet 143 provides for air to be added or released from the balloon 140.

FIG. 11b is a diagram illustrating an example of a perspective view of the apparatus 100 illustrated in FIG. 11a. FIG. 11c is diagram illustrating an example of the apparatus of FIGS. 11a and 11b, but from a different perspective view. FIG. 11d is a diagram illustrating an example of front view of the apparatus illustrated in FIGS. 11a-11c

VII. Process Flow View

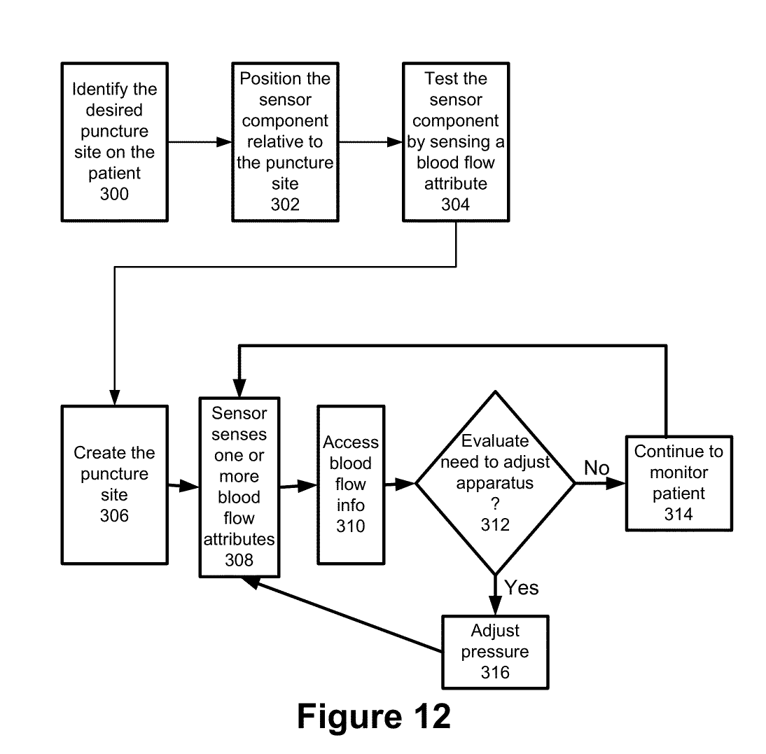

FIG. 12 is a flow chart diagram illustrating an example of a processing for using the hemostasis sensor apparatus 100 to perform hemostasis on a patient 90.

At 300, a desired puncture site 89 (i.e. location) is identified on the patient 90.

At 302, position the sensor component 110 relative to the puncture site 89. This step can be performed before the puncture site 89 is created at 306, or after.

At 304, test the sensor component 110 by sensing one or more blood flow attributes 87. In many embodiments, it is desirable for this step to be performed before the puncture site 89 is created at 306.

At 306, the puncture site 89 is created by the provider 92. This step triggers the need to initiate hemostasis.

At 308, the sensor component 110 is used to sense information relating to the blood flow of the patient 92 (i.e. blood flow attributes 87).

At 310, the provider 92 accesses the information captured by the sensor apparatus 100.

At 312, the provider 92 evaluates the need to adjust the pressure being applied to the puncture site 89 as part of the hemostasis process.

If the sensed blood flow attributes 87 indicate that an adjustment in the pressure applied to the puncture site 89 is warranted, then the pressure is adjusted at 316. Otherwise, at 314 the apparatus 100 continues to monitor the patient 90 through the completion of the hemostasis process. As indicated in FIG. 12, there is a processing loop beginning with the initial capture of one or more blood flow attributes 308 through the completion of the hemostasis process.

VIII. Index of Elements

Table 1 below includes a table listing certain element numbers, element names, and a brief description of the applicable element.

| Element | | |
|---|---|---|
| Number | Name | Description |
| 87 | Blood Flow | An attribute of the patient 90 that relates to blood flow at or |
| | Attribute | around the puncture site 89. Blood flow attributes are input |
| | | parameters to the sensor apparatus 100. Different sensor |
| | | components 110 can capture blood flow information in different |
| | | formats and at different degrees of precision. |
| 88 | Sensor Reading | An output related to blood flow generated by the sensor |
| | | apparatus 100. The communication component 111 of the |
| | | sensor apparatus 100 provides the provider 92 with access to |
| | | blood flow information. Different communication components |
| | | 111 can communicate blood flow information in different formats |
| | | and at different degrees of precision. |
| 89 | Puncture Site | A location on the patient 90 for which hemostasis is performed |
| | | to prevent blood loss. In the context of catheterizations, the |
| | | term “arteriotomy” is synonymous with the term “puncture site”. |
| 90 | Patient | A living organism, typically a human being, subject to a |
| | | hemostasis process. |
| 91 | Radial Artery | A blood vessel on a human patient 90 used to perform radial |
| | | catheterization. The puncture site 89 for a radial catheterization |
| | | procedure is a location on Radial Artery 91 near a patient's 90 |
| | | wrist. |
| 92 | Provider | A doctor, nurse, nurse practitioner, catheterization lab |
| | | technician, physician assistant, paramedic, or other person |
| | | involved in performing hemostasis on a patient |
| 100 | Sensor | An assembly of components that includes a sensor component |
| | Apparatus (or | 110 and a communication component 111. The apparatus 100 |
| | simply the | is used to monitor the blood flow of a patient 90 at or around the |
| | Apparatus) | puncture site 89 during the performance of hemostasis. |
| 102 | Body | A band or other component or surface that can be used to hold |
| | Component | together various components of a fully or partially integrated |
| | | sensor apparatus 100. A body component 102 can be fully |
| | | flexible, fully rigid, or semi-rigid/semi-flexible. Examples of body |
| | | components 102 include a flexible band 130, a strip 131, a loop |
| | | 132, a semi-flexible/semi-rigid band 134, and a fully rigid band. |
| 104 | Fastening | A mechanism to secure the position of the body component 102 |
| | Component | or the sensor apparatus 100 to a position on the patient 90 |
| | | relative to the puncture site 89. |
| 106 | Padding | A mechanism to enhance the comfort of the patient 90 with |
| | Component | respect to the apparatus 100. |
| 108 | Pressure | Potentially any mechanism used to apply pressure on the |
| | Component | puncture site 89, including but not limited to a balloon 140. |
| 110 | Sensor | A component or subassembly within the sensor apparatus 100 |
| | Component | that captures a sensor reading 88 from the patient 90 relating to |
| | | blood flow of the patient 90. Categories of potential sensor |
| | | components 110 include but are not limited to electronic sensors |
| | | 160, mechanical sensors 162, electro-mechanical sensors 164, |
| | | and films 120 such as reflective foils 121. |
| 111 | Communication | A component or subassembly within the sensor apparatus 100 |
| | Component | that communicates the sensor reading 88 outside the sensor |
| | | apparatus 100 so that the information can be acted upon by a |
| | | provider 92 |
| 112 | Timer | Potentially any mechanism on the apparatus 100 or in |
| | Component | communication with the apparatus 100 that is used to: (1) |
| | | measure the passage the time in providing hemostasis to the |
| | | patient 90; (2) evaluate/adjust sensor readings 88 in the context |
| | | of how long the hemostasis process has been performed on the |
| | | patient 90; and/or (3) determine the frequency and/or duration of |
| | | time for which sensor readings 88 are captured. |
| 113 | Exterior System | An information technology system 113 used to treat the patient |
| | | 90 that is outside the scope of the sensor apparatus 100. The |
| | | sensor readings 88 of the sensor apparatus 100 can be |
| | | integrated into broader and/or comprehensive systems being |
| | | used to treat the patient 90. |
| 114 | Sensor | A mechanism or component used to enhance the ability of the |
| | Enhancement | sensor component 110 to capture sensor readings 88. For |
| | Component | example, a “middle member” or “air pocket” of a balloon 140 |
| | | underneath a reflective foil 121 can enhance the ability of the |
| | | reflective foil 121 to visually convey the pulsing motion at or |
| | | around the puncture site 89. |
| 115 | Adhesive | A component, coating, or layer used to secure the position of |
| | Component | the sensor component 110 or the entire sensor apparatus 100 |
| | | with respect to the patient 90 or other components of the sensor |
| | | apparatus 100. For stand-alone embodiments of the sensor |
| | | apparatus 100, an adhesive layer on the apparatus 100 can |
| | | secure the position of the apparatus 100 on the patient 90 |
| | | without the use of a band. |
| 116 | Anti-Adhesion | A component, coating, or layer used to prevent some surface of |
| | Component | the apparatus 100 from sticking to the patient 90. Pressure |
| | | components 108 may in particular benefit from an anti-adhesion |
| | | component such as a medical grade silicone coating. |
| 117 | Computer | A processing unit that implement programming logic within the |
| | | apparatus 100. In some embodiments of the apparatus 100, the |
| | | computer 117 within the apparatus 100 is also the |
| | | communications component 111 by which sensor readings 88 |
| | | are conveyed outside the apparatus 100. |
| 119 | Transmitter | A communications component 111 based on the transmission of |
| | | electromagnetic signals. Transmitters 119 can be wired as well |
| | | as wireless. |
| 120 | Film | A substrate with sufficient flexibility to function as a sensor |
| | | component 110 by fluctuating in shape and/or position in a |
| | | responsive manner to the blood flow pulses of the patient 90 |
| | | during hemostasis. |
| 121 | Reflective Foil | A sub-category of film 120 that functions as both a sensor |
| | | component 110 and a communications component 111 in that a |
| | | reflective foil 120 can visually convey blood flow information by |
| | | changes in reflective light from the foil 121. |
| 122 | Pattern | A visual model, archtype, or arrangement that can be displayed |
| | | on a reflective foil 121 to convey blow flow information relating |
| | | to the patient 90. A pattern 122 can also be referred to as a |
| | | hologram. |
| 124 | Sheath | A transparent or substantially transparent cover over film 120 |
| | | such as reflective foil 121. |
| 125 | Window | A transparent area within reflective foil 121 that permits the |
| | | provider to see the puncture site 89. |
| 126 | Timer | An example of a timing component 112 |
| 127 | Zones | An individual area of space on a grid 129. |
| 128 | Ink Release | An example of a timer component 112. |
| | Strip | |
| 129 | Grid | A matrix of zones 127 that can collectively comprise the surface |
| | | area of a film 120. In many embodiments, a 2×2 or 3×3 grid 129 |
| | | is desirable. |
| 130 | Flexible Band | An example of a body component 102. |
| 131 | Strip | An example of a body component 102. |
| 132 | Loop | An example of a body component 102. |
| 134 | Semi-Flexible | An example of a body component 102. |
| | Band | |
| 136 | Pad | An example of a pressure component 108 and/or padding |
| | | component 106. |
| 140 | Balloon | A balloon 140 can function as a padding component 106, a |
| | | pressure component 108, and/or as a sensor enhancement |
| | | component 114. The flexibility of a balloon 140 can be helpful to |
| | | the sensor component 110 in amplifying information relating to |
| | | the blood flow of the patient 90. Balloons 140 can provide for |
| | | inflation and deflation in a variety of different ways. |
| 142 | Pulse Gauge | An example of a communications component 111 in which |
| | | sensor readings 88 are display in either a numerical format, or |
| | | some other form of linear scale. |
| 143 | Inlet | An opening into a balloon 140 which provide for the inflation or |
| | | deflation of the balloon 140. |
| 144 | Display Screen | An electronic device capable of displaying data, text, or other |
| | | images. A display screen 144 is an example of a |
| | | communications component 111. |
| 145 | Gaps | An opening within the balloon 140 that allows for the sliding of |
| | | the balloon 140 along the body component 102. |
| 150 | Piston Assembly | An example of a mechanical sensor component 162. |
| 152 | Floating Piston | A component in a piston assembly 152 that moves within an |
| | | oval cylinder 154 in response to the pulse of the patient 90. |
| 154 | Oval Cylinder | A component in a piston assembly 150 within which a floating |
| | | piston 152 can move. |
| 156 | Inflation Tube | An air intake passageway for the piston assembly 150. |
| 160 | Electronic | A category of sensor component 110 in which the sensor |
| | Sensor | component 110 captures sensor readings 88 through the use of |
| | | electricity |
| 162 | Mechanical | A category of sensor component 110 in which the sensor |
| | Sensor | component 110 captures sensor readings 88 through the use |
| | | mechanical properties and functions. |
| 164 | Electro- | A category of sensor component 110 that is both a mechanical |
| | Mechanical | sensor 162 and an electronic sensor 160. |
| | Sensor | |
| 166 | Doppler Sensor | An electronic sensor 160 that captures sensor readings 88 |
| | | based on the principle that sound pitch increases as the source |
| | | moves toward the listener and decreases as it moves away |
| 168 | Ultrasound | An electronic sensor 160 that captures sensor readings 88 |
| | Sensor | based on the principle that sound reflected back from an aimed |
| | | target can be detected back at is origin |

1. A hemostasis sensor apparatus for use by a provider to stop the bleeding from a puncture site of a patient, said hemostasis sensor apparatus comprising:
a sensor component positioned with respect to the puncture site, said sensor component providing for the capture of a sensor reading relating to a blood flow attribute of the patient; and
a communication component, said communication component providing for the communication of said sensor reading to the provider.

2. The hemostasis sensor apparatus of claim 1, wherein said sensor component is an electronic sensor.

3. The hemostasis sensor apparatus of claim 1, wherein said sensor component is a mechanical sensor.

4. The hemostasis sensor apparatus of claim 1, wherein said sensor reading is a visual display relating to said blood flow attribute of the patient.

5. The hemostasis sensor apparatus of claim 1, wherein said sensor component is integral with said communication component and wherein said sensor component does not provide for being separated from said communication component.

6. The hemostasis sensor apparatus of claim 1, wherein said sensor component includes a balloon in direct contact with the patient.

7. The hemostasis sensor apparatus of claim 1, said hemostasis apparatus further comprising a reflective foil, wherein said reflective foil is said sensor component and said communication component, wherein said puncture site relates to a radial artery, and wherein the patient is a human being,

8. The hemostasis sensor apparatus of claim 7, wherein said reflective foil is a hologram foil and wherein said reflective foil comprises a grid of at least four zones.

9. The hemostasis sensor apparatus of claim 1, wherein said sensor component provides for being positioned on the patient before the puncture site is created.

10. The hemostasis sensor apparatus of claim 1, wherein said sensor component includes at least one of: (a) a Doppler sensor and (b) an ultrasound sensor.

11. The hemostasis sensor apparatus of claim 1, said hemostasis sensor apparatus further comprising a body component connected to said sensor component and said communication component.

12. The hemostasis sensor apparatus of claim 1, wherein said body component is a substantially flexible and substantially transparent band, and wherein said sensor component is not positioned on the puncture site.

13. The hemostasis sensor apparatus of claim 1, said hemostasis sensor apparatus further comprising a pressure component.

14. The hemostasis sensor apparatus of claim 1, said hemostasis sensor apparatus further comprising a padding component.

15. The hemostasis sensor apparatus of claim 1, said hemostasis sensor apparatus further comprising a fastening component.

16. A hemostasis sensor apparatus for use by a provider to stop the bleeding from a puncture site of a patient, said hemostasis sensor apparatus comprising:
a sensor component positioned with respect to the puncture site, said sensor component providing for the capture of a sensor reading relating to the puncture site;
a communication component, said communication component providing for the communication of said sensor reading to the provider;
a body component connected to said sensor component and to said communication component;
a fastening component that provides for securely positioning said hemostasis apparatus on the patient with respect to the puncture site; and
a pressure component that provides for the application of pressure on the puncture site.

17. The hemostasis sensor apparatus of claim 16, wherein said sensor component is integral with said communication component.

18. The hemostasis sensor apparatus of claim 16, said hemostasis sensor apparatus further comprising a plurality of balloons, said plurality of balloons including a first balloon and a second balloon, wherein said sensor component includes said first balloon and wherein said pressure component includes said second balloon.

19. The hemostasis sensor apparatus of claim 16, wherein said communication component includes a visual display that is comprised of a nine-zone grid.

20. A method for using a hemostasis sensor in the performance of hemostasis on a puncture site of a patient, comprising:
positioning the hemostasis sensor apparatus relative to the puncture site;
creating the puncture site;
access a sensor reading captured using the hemostasis sensor apparatus; and
adjust the pressure applied to the puncture site in response to the accessed sensor reading.
